SEQUENCE LISTING (14 sequences)

SEQ ID NO: 1 - DNA, 4203 bp, Artificial Sequence
  Description of Artificial Sequence Synthetic polynucleotide
atggccccaaagaaaaagcggaaggtcggtatccacggagtcccagcagccgacaagaaatacagcatcggcctggacatcggcaccaactctgtgggctgggccgtgatcaccgacgagtacaaggtgcccagcaagaaattcaaggtgctgggcaacaccgacagacacagcatcaagaagaacctgatcggagccctgctgttcgacagcggcgaaacagccgaggccacccggctgaagagaaccgccagacggagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaaaagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaagccccactgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagagaatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaacaggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaaactgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccatcacgcccatgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcataagcactacctggacgagatcatcgagcagatcagcgagttctccaagagagtgatcctggccgacgctaatctggacaaagtgctgtccgcctacaacaagcatcgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacaagcgtcctgctgctactaagaaagctggtcaagctaagaaaaagaaatga

SEQ ID NO: 2 - DNA, 4203 bp, Artificial Sequence
  Description of Artificial Sequence Synthetic polynucleotide
atggccccaaagaaaaagcggaaggtcggtatccacggagtcccagcagccgacaagaaatacagcatcggcctggccatcggcaccaactctgtgggctgggccgtgatcaccgacgagtacaaggtgcccagcaagaaattcaaggtgctgggcaacaccgacagacacagcatcaagaagaacctgatcggagccctgctgttcgacagcggcgaaacagccgaggccacccggctgaagagaaccgccagacggagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaaaagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaagccccactgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagagaatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaacaggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaaactgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccatcacgcccatgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcataagcactacctggacgagatcatcgagcagatcagcgagttctccaagagagtgatcctggccgacgctaatctggacaaagtgctgtccgcctacaacaagcatcgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacaagcgtcctgctgctactaagaaagctggtcaagctaagaaaaagaaatga

SEQ ID NO: 3 - DNA, 4203 bp, Artificial Sequence
  Description of Artificial Sequence Synthetic polynucleotide
atggccccaaagaaaaagcggaaggtcggtatccacggagtcccagcagccgacaagaaatacagcatcggcctggacatcggcaccaactctgtgggctgggccgtgatcaccgacgagtacaaggtgcccagcaagaaattcaaggtgctgggcaacaccgacagacacagcatcaagaagaacctgatcggagccctgctgttcgacagcggcgaaacagccgaggccacccggctgaagagaaccgccagacggagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaaaagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaagccccactgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagagaatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaacaggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaaactgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccatcacgcccatgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcataagcactacctggacgagatcatcgagcagatcagcgagttctccaagagagtgatcctggccgacgctaatctggacaaagtgctgtccgcctacaacaagcatcgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacaagcgtcctgctgctactaagaaagctggtcaagctaagaaaaagaaatga

SEQ ID NO: 4 - DNA, 4203 bp, Artificial Sequence
  Description of Artificial Sequence Synthetic polynucleotide
atggccccaaagaaaaagcggaaggtcggtatccacggagtcccagcagccgacaagaaatacagcatcggcctggccatcggcaccaactctgtgggctgggccgtgatcaccgacgagtacaaggtgcccagcaagaaattcaaggtgctgggcaacaccgacagacacagcatcaagaagaacctgatcggagccctgctgttcgacagcggcgaaacagccgaggccacccggctgaagagaaccgccagacggagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaaaagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaagccccactgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagagaatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaacaggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaaactgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccatcacgcccatgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcataagcactacctggacgagatcatcgagcagatcagcgagttctccaagagagtgatcctggccgacgctaatctggacaaagtgctgtccgcctacaacaagcatcgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacaagcgtcctgctgctactaagaaagctggtcaagctaagaaaaagaaatga

SEQ ID NO: 5 - DNA, 44 bp, Artificial Sequence
  Description of Artificial Sequence Synthetic primer
  modified_base (27)..(44): a, c, t, g, unknown or other
gagtaagaagaaatataagagccaccnnnnnnnnnnnnnnnnnn

SEQ ID NO: 6 - DNA, 41 bp, Artificial Sequence
  Description of Artificial Sequence Synthetic primer
  modified_base (24)..(41): a, c, t, g, unknown or other
aggcaagccccgcagaaggcagcnnnnnnnnnnnnnnnnnn

SEQ ID NO: 7 - DNA, 23 bp, Artificial Sequence
  Description of Artificial Sequence Synthetic primer
gctgccttctgcggggcttgcct

SEQ ID NO: 8 - DNA, 26 bp, Artificial Sequence
  Description of Artificial Sequence Synthetic primer
ggtggctcttatatttcttcttactc

SEQ ID NO: 9 - DNA, 120 bp, Artificial Sequence
  Description of Artificial Sequence Synthetic primer
tttttttttttttttttttttttttttttttttttttttttttttttttttttttttttttttttttttttttttttttttttttttttttttttttttttttttttttttttttttttt

SEQ ID NO: 10 - DNA, 23 bp, Artificial Sequence
  Description of Artificial Sequence Synthetic oligonucleotide
tcagcatctttccatttcactgg

SEQ ID NO: 11 - DNA, 23 bp, Artificial Sequence
  Description of Artificial Sequence Synthetic oligonucleotide
tcagcatctttccatttcactgg

SEQ ID NO: 12 - DNA, 23 bp, Artificial Sequence
  Description of Artificial Sequence Synthetic oligonucleotide
gggtaagataaggagagttcgga

SEQ ID NO: 13 - DNA, 23 bp, Artificial Sequence
  Description of Artificial Sequence Synthetic oligonucleotide
ggggtgtttcttgggaaacgttt

SEQ ID NO: 14 - DNA, 17 bp, Artificial Sequence
  Description of Artificial Sequence Synthetic oligonucleotide
aaacagggaagggaaga

(19) United States
(12) Patent Application Publication
(10) Pub. No.: US 2022/0195403 A1
(43) Pub. Date: Jun. 23, 2022

(54) METHODS OF ACHIEVING HIGH SPECIFICITY OF GENOME EDITING

(71) Applicant: ALLELE BIOTECHNOLOGY AND PHARMACEUTICALS, INC., San Diego, CA (US)

(72) Inventors: Jiwu WANG, La Jolla, CA (US); Andrew M. CHAMMAS, Escondido, CA (US); Alexander WARD, Del Mar, CA (US)

(21) Appl. No.: 17/259,998

(22) PCT Filed: Jul. 12, 2019
(86) PCT No.: PCT/US2019/041551

§ 371 Date: Jan. 13, 2021

Related U.S. Application Data
(60) Provisional application No. 62/697,955, filed on Jul. 13, 2018.

(51) Int. Cl. C12N 9/22; C12N 15/11; C12N 15/90; C12N 15/115

(52) U.S. Cl. CPC C12N 9/22; C12N 15/11; C12N 15/907; C12N 2310/3519; C12N 2310/20; C12N 2800/80; C12N 2310/16; C12N 15/115

(57) ABSTRACT

A method is disclosed for highly efficient DNA sequence alterations. The method is useful for editing chromosomes, to engineer cellular markers through insertion of genes, or to create epigenetic changes by using cas9-enzyme fusions where the enzymes can be DNA epigenetic modifying enzymes or chromatin modifying enzymes, etc. The technology also differs from all previously known technologies in that the CRISPR/Cas system can function in ways that are “clean”, i.e. they have not been in contact with any virus, or are carried DNA molecules that can insert into the chromosome in unintended locations.

### RELATED APPLICATIONS

This application claims the benefit of priority to U.S. Ser. No. 62/697,955, filed on Jul. 13, 2018, which is incorporated herein in its entirety, including the drawings.

### SEQUENCE LISTING

The instant application contains a Sequence Listing which has been submitted electronically in ASCII format and is hereby incorporated by reference in its entirety. Said ASCII copy, created on Aug. 5, 2019, is named F6035-00136_SL.txt and is 25,646 bytes in size.

### FIELD OF THE INVENTION

This disclosure relates to methods, compositions, and kits and systems that can be used in DNA modification, including DNA sequence knock-in or knock-out, DNA mutation, DNA epigenetic modification, chromatin modification in a DNA sequence-specific manner, and other types of genome editing. More specifically, this disclosure relates to methods that can deliver the system of Clustered Regularly Interspaced Short Palindromic Repeats (CRISPR) and components, mutations, fusions, and variations thereof, without the use of any carrier vector. This invention specifically teaches a process of editing a genome with specificity and precision that permits substituting a single nucleotide, including host cells as challenging as a pluripotent stem cell.

### BACKGROUND OF THE DISCLOSURE

The previously reported CRISPR/CAS studies in cultured mammalian cells relied on DNA vectors or retrovirus/lentivirus for delivering both the sgRNA and the Cas enzyme for example, see U.S. Pat. No. 8,697,359. Plasmid DNA presents the possibility of random DNA integration into the host genome, which is widely known in the art (for instance, see Valamehr et al. 2014 Stem Cell Reports). The retroviral or lentiviral vectors for delivery of the cas enzyme genes or gRNA need to integrate into the host genome before they can deliver the payload they carry as RNA or protein molecules. Additionally, it is difficult to control the level of expression from either plasmid or viral vectors. Even though there is a general correlation between the expression level of encoded genes on these vectors and the copy number of the vectors, the relationship is non-linear and highly variable.

### SUMMARY OF THE INVENTION

A novel method is disclosed for highly efficient DNA sequence alterations. The method can be used to edit chromosomes, to engineer cellular markers through insertion of genes, or to create epigenetic changes by using cas9-enzyme fusions where the enzymes can be DNA epigenetic modifying enzymes or chromatin modifying enzymes, etc. In addition to the dramatically increased efficiency of genome editing by the invented process, the novel technology also differs from all previously known technologies in that the CRISPR/CAS system can function in ways that are “clean”, i.e. they have not been in contact with any virus, or are carried DNA molecules that can insert into the chromosome in unintended locations. It is also noted that the disclosed system can generate previously unattainable efficiency while keeping off-target changes to the minimum. Utility of the invention can be found in virtually all areas that involve DNA editing or epigenetic modification. In contrast, the 8,697,359 patent does not teach how to provide a system where CRISPR/Cas can be efficiently attained in eukaryotic cells while minimizing the potential problem of unintended genome changes.

The current disclosure provides an RNA-based system that provides both the Cas enzymes and guide RNAs, and in cases involving DNA break repair, a “patch” template RNA or DNA, all without the need of any exogenous DNA molecules (except when a DNA template is a preferred template for DNA break repair). The all-RNA CRISPR/Cas (as used herein, the term “all-RNA” primarily refers to the delivery of the components of a CRISPR/Cas machinery and does not exclude DNA as template) system disclosed herein does not require any viral elements that may create problems for human clinical use of the process or resulting cells. This system can be provided as methods, processes, or reagent kits to achieve gene disruption through CRISPR/Cas-promoted indels, genome sequence editing to the precision of a single base, or gene replacement through break repair and replace after CRISPR/Cas treatment in cultured cells, including embryonic stem cells (ESCs) and induced pluripotent stem cells (iPSCs), at enhanced efficiency and specificity compared to what has been shown in the field.

An important aspect of the current disclosure is the use of an all-RNA delivery method to enable a polynucleotide-guided genomic cutting system in eukaryotic cells, with designs particularly useful in mammalian cells, and a process empirically developed for difficult-to-maintain cells such as pluripotent stem cells, which easily escape the pluripotency state if perturbed. The disclosed method will also work in tissue stem cells such as, without limitation, neural progenitor cells, oligodendrocyte progenitor cells, mesenchymal stem cells, hematopoietic stem cells, etc. Provided herein are methods that introduce the gRNA as in vitro transcribed (IVT) RNA and the Cas enzyme as mRNA using common nucleoside triphosphates (NTP) or NTP with chemical modifications.

Another aspect of disclosure in addition to being footprint-free (unlike plasmid vectors that can integrate into the genome), is that using RNA as the delivery format enables higher enzyme activity level of Cas which results in higher success rates. In another disclosure, the high level of enzyme activity can be concentrated within a short-time window in a highly controllable fashion. The transient nature of RNA-mediated high enzyme expression level provides an ideal composition for the purpose of chromosomal modification. The short-burst enzyme expression provides additional benefits in reducing off-target effects because long existence of the enzyme, such as that from plasmid DNA vectors or integrated viral vectors can result in continued off-target effects.

In another aspect of the disclosure, the gRNA is delivered at various ratios to Cas mRNA, sometimes involving multiple delivery via transfection. Because once mRNA of cas is translated into Cas protein, the protein is likely to have a longer half-life than mRNAs and gRNA. The disclosure herein demonstrates that, by adjusting the gRNA amount, which can also be referred to as gRNA/cas mRNA ratio, the process can result in precise, single-base editing, in addition to more commonly seen longer inserts or deletions or rearrangements of the chromosome. Example 4 of the current disclosure demonstrates the increased precision of the disclosed methods by showing a successful example of how a single base on the chromosome can be changed using the all-RNA methodology in a human iPSC clone.

A stumbling block to using mRNA to achieve prolonged protein expression in cell culture is that the RNA itself can be highly immunogenic (Kawai and Akira, 2007; Randall and Goodbourn, 2008). Mammalian cells are equipped with a battery of sensors that can detect exogenous RNA and activate antiviral defense pathways which prime cytostatic and apoptotic pathways and alert neighboring cells to the very same stimuli via excreted signals such as interferon alpha and beta. The more broadly-expressed sensors such as TLR3 and RIG-I primarily detect double-stranded RNA (the production of dsRNA being a distinctive feature in many viral life cycles) but can also be activated by synthetic mRNA (Kormann et al., 2011). Technical means were found to minimize immunogenic responses to synthetic mRNA during the course of iPSC generation with mRNA (Warren et al., 2010). The most practical approaches involved the incorporation of modified nucleobases and supplementation of culture media with a recombinant version of B18R protein when treating human cells with modified mRNAs, an extracellular decoy receptor for Type I interferons naturally expressed by Vaccinia virus to blunt immune responses to infection.

In one embodiment, the delivery of the all-RNA CRISPR/Cas system into human cells was accompanied with the addition of B18R. In another embodiment, the RNA molecules can be delivered into human or non-human cells when the RNA molecules are sufficiently purified to remove aberrant transcripts during in vitro transcription. In another embodiment, the delivered RNA molecules are modified to evade cellular immune detection. In summary, the novel CRISPR/Cas system provides technical enablement for genome engineering in these aspects: polynucleotide-guided, without the requirement of protein engineering for each target site; fully controlled process through RNA delivery that does not leave a genomic footprint; easy to achieve desired modification efficiency in different cell types by varying treatment time; higher success rate and lower off-target effects than ZFN or TALEN or previously reported CRISPR/CAS methodologies because of the designed higher enzyme activity in a shorter time window; precise genome modification in a highly efficient process that can be performed in pluripotent stem cells without perturbing the stem cell state, enabled at least in part by a previously unknown and nearly uncontrollable factor—the gRNA/cas-mRNA ratio, which is not optimal if plasmids, viral vectors, and ribonucleoprotiens (RNPs) are used. Compared to recently published CRISPR/CAS systems, the disclosed all-RNA format uniquely enables minimization of unwanted chromosomal changes.

The method disclosed herein is based on the unexpected benefits of adjusting doses of gRNA and CAS enzyme via cas mRNA. We disclose the time and frequency of delivery, and method for delivery into human cells and by simple expansion, any mammalian cells; using a similar scheme, the CRISPR/CAS system described herein can also be used in other types of cells, such as those of plants, yeasts, bacteria.

In embodiments of the aspects of the disclosure discussed above, disclosed herein are methods for genome editing that uses a combination of synthetic mRNA that encodes Cas9 enzyme and sgRNA. In embodiments of this aspect, the mRNA that encodes Cas9 and sgRNA contains a 5′diguanosine cap and poly(A) tail, and modified nucleotides that make the mRNA less toxic to a cell. In some embodiments, the modified nucleotides comprise 5-methyl-Cytosine, 2-Thio-Uracil, or pseudouracil. In some embodiments, the mRNA encoding Cas9 is given together with B18R.

In another aspect of the disclosure, disclosed herein are methods for making precise changes to DNA or the genome using mutated forms of Cas9 protein that contain mutations to one or both of their endonuclease genes. In embodiments of this aspect, Applicant have produced three non-naturally occurring mutant Cas9 proteins with mutations to their endonuclease active site. These mutant Cas9 proteins are encoded by SEQ ID NOS: 2, 3, and 4.

Another aspect of the disclosure are methods that enable very precise repair of point mutations that are based on the use of the mutated Cas9 proteins. In one embodiment, a non-naturally occurring CRISPER-Cas system comprising an mRNA that encodes for a mutated Cas9 protein that has a mutation in its endonuclease active site and at least one mRNA that encodes for a guide RNA that upon entry into a cell produces the mutated Cas9 protein and guide RNA. After entry, the Cas9 protein and guide RNA targets and hybridizes to a target sequence of a DNA with a single point mutation that upon action of the mutant Cas9 protein and guide RNA corrects the mutation in the target sequence.

In an embodiment, disclosed herein are methods for genome editing that uses a combination of synthetic mRNA that encodes Cas9 enzyme and sgRNA. In some embodiments the mRNA that encodes Cas9 and sgRNA contains a 5′diguanosine cap and poly(A) tail. In some embodiments, a template to facilitate DNA break is also provided. The template can be a double-stranded DNA molecule or single-stranded DNA molecule. In some embodiments, the template is an RNA molecule. In an embodiment of this method, the Cas9 bears a mutation that disrupts one of the two endonuclease active sites. The Cas9 protein mutants are encoded by SEQ ID NO: 2, or SEQ ID NO: 3. One Cas9 protein mutant has mutations in both endonuclease active sites and is encoded by SEQ ID NO: 4. In another embodiment of the method, Cas9 is fused to another enzyme that can alter epigenetic markers on either the DNA or chromatin proteins. In some embodiments of the method, the molar ratio between Cas9 mRNA:sgRNA is between 1:1,000 to 1,000:1. In some embodiments of the method, the molar ratio between Cas9 mRNA:sgRNA is between 1:1,000 to 1,000:1. In some embodiments the molar ratio of Case9mRNA:sgRNA is 1:1,000; 1:950; 1:900; 1:850; 1:800; 1:750; 1:700; 1:650; 1:600; 1:550; 1:500, 1:450; 1:400; 1:350; 1:300; 1:250; 1:200; 1:150; 1:100; 1:50; 1:40; 1:30; 1:25; 1:20; 1:15; 1:10; 1:9; 1:8; 1:7; 1:6; 1:5; 1:4.75; 1:4.5; 1:4.25; 1:4; 1:3.75; 1:3.5; 1.3.25; 1:3; 1:2.9; 1:2.8; 1:2.75; 1:2.7; 1:2.6; 1:2.5; 1:2.4; 1:2.3; 1:2.25; 1:2.2; 1:2.1; 1:2; 1:1.9; 1:1.8; 1:1.7; 1:1.6; 1:1.5; 1:1.4; 1:1.3; 1:1.2; 1:1.1; 1:1; 1.1:1; 1.2:1; 1.3:1; 1.4:1; 1.5:1; 1.6:1; 1.7:1; 1.8:1; 1.9:1; 2:1; 2.1:1; 2.2:1; 2.25:1; 2.3:1; 2.4:1; 2.5:1; 2.6:1; 2.7:1; 2.75:1; 2.8:1; 2.9:1; 3.0:1; 3.25:1; 3.5:1; 3.75:1; 4:1; 4.25:1; 4.5:1; 4.75:1; 5:1; 6:1; 7:1; 8:1; 9:1; 10:1; 15:1; 20:1; 25:1; 30:1; 40:1; 50:1; 100:1; 150:1; 200:1; 250:1; 300:1; 350:1; 400:1; 450:1; 500:1; 550:1; 600:1; 650:1; 700:1; 750:1; 800:1; 850:1; 900:1; 950:1; 1,0000:1, or any range of ratios between any two of the recited ratios. In another embodiment, multiple sgRNAs targeting different sites in combination with mRNA molecules encoding one or more different Cas9 enzymes from different species or bearing different mutations are introduced into the same cells. In the method disclosed herein, the repair template is localized to the DNA break site through fusion to the sgRNA as on one molecule In some embodiments, the repair template is localized to the DNA break site through fusion to an aptamer that binds Cas9.

The precision-enabling nature of the disclosed methods makes the disclosed technology most suitable for creating cells for treating human diseases, such as without limitation, Methylmalonyl-CoA mutase deficiency, 3-Methylcrotonyl-CoA carboxylase deficiency, Gaucher's disease, Ogden syndrome, Lesch-Nyhan syndrome, Leigh disease, pyruvate dehydrogenase deficiency, 3-hydroxy-3-methylglutaryl-CoA lyase deficiency, carboxylase deficiency, multiple, late-onset, fumarase deficiency, fibrodysplasia ossificans progressive, n-glycanse 1 deficiency, siderius type X-linked mental retardation, phenylketonuria, tay-sachs disease, alpha-galactosidase A deficiency, sickle cell anemia, maple syrup urine disease.

### BRIEF DESCRIPTION OF THE DRAWINGS

The invention will now be described in relation to the drawings in which:

FIG. 1. Creation of IVT templates for generating cas9 mRNA sgRNA 2% agarose gel shows bands of purified linearized DNA generated by cutting cas9 or sgRNA gene encoding plasmids with restriction enzymes.

FIG. 2. mRNA encoding the Cas9 enzyme and sgRNA against fluorescent protein mWasabi. 2% agarose gel shows band of the cas9 mRNA with poly(A) tails and the sgRNA against mWasabi.

FIG. 3. Effects of disrupting the expression of mWasabi gene integrated in to the chromosomes of human 293 cells. Constant amount of cas9 mRNA and increasing amount of sgRNA was delivered into 293-mWasabi cells in a single transfection. The control well did not receive any RNA but was treated with the same transfection reagents.

FIG. 4. Examples of using all-RNA CRISPR/CAS system in creating a mutation in human gene. Each dsDNA break point can be directed by a pair of sgRNAs. A sequence replacement can be made with either one or two break points as shown in the figure. When 4 sgRNAs are relied upon to direct the replacement, the specificity is maximized. FIG. 4 discloses SEQ ID NOS 10-13, respectively, in order of appearance.

FIG. 5. Examples of using all-RNA CRISPR/CAS system in creating a mutation in human gene with a dimerized Cas9 enzyme encoded by modified mRNA. The CRISPR/CAS mediated genome editing specificity can be further enhanced with dimerizing Cas9, particularly when delivered through encoding mRNA. Other domains can be fused to Cas9 in a similar fashion for epigenetic modifications.

FIG. 6. Primer design for qPCR. This design enabled detection of a single base change in iPSCs by real time PCR

FIG. 7. Example of Amplification Ct curves. This curve shows how mutation rate at a given location on the chromosome was detected by well-designed qPCR.

FIG. 8. Sample amplification plot for clonal amplicon library screening. qPCR screening of clonal amplicon libraries commonly result in high variation, however given a bulk population that has an HDR efficiency of ˜1%, there will be a small number of low Ct outlier wells. Once left shifted Ct outliers were identified, and the corresponding wells were expanded in duplicate plate.

FIG. 9. Sample chromatograms for clonal amplicon library screening. A single base switch from T to G was achieved in a single iPSC clone which is heterozygous for the intended MEF2C locus. FIG. 9 discloses SEQ ID NO: 14.

### DETAILED DESCRIPTION OF THE INVENTION

When describing the present invention, all terms not defined herein have their common meanings recognized in the art. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention.

Other workers in the field have tried to use in vitro transcribed cas mRNA and gRNA, but with no success or limited results. For example, Kouranova et al. (Hum Gene Ther. 2016 Jun. 1; 27(6): 464-475.) tried plasmid, RNA, and protein as the delivery format of Cas in comparison with ZFN. They concluded that “Unlike our experience with ZFN mRNAs, co-transfection of in vitro-transcribed Cas9 mRNA or Cas9-expressing plasmid DNA with in vitro-transcribed sgRNAs rarely led to efficient cleavage at the target sites in the rat C6 cell line by nucleofection”. Liang et al. (Journal of Biotechnology, Volume 208, 20 Aug. 2015, 44-53), also compared plasmid, mRNA, and protein for delivering CRISPR/CAS into various mammalian cells. Whereas they demonstrated that both mRNA and RNP-forming CAS protein worked in creating indels, homology directed recombination (HDR), which is the mechanism for precisely editing a base on the chromosome, a much more difficult task and often more desired outcome through CRISPR/CAS, was not performed or presented, and highly unlikely to be achieved in their system. Others have used RNA molecules for CRISPR/CAS, but only in fertilized animal eggs or embryos through microinjection, to various results (Wu et al. Cell Stem cell, Volume 13, Issue 6, 5 Dec. 2013, 659-662; Liang et al. Protein & Cell, May 2015, Volume 6, Issue 5, pp 363-372; Hruscha et al. Development 2013 140: 4982-4987). None of these reports were based on a transfection process as disclosed herein that is successful for use with mammalian cell cultures maintained in a vessel, including particularly difficult-to-maintain cells such as pluripotent stem cells.

In one aspect of the current disclosure, mRNA-based encoding wild-type cas9 from different bacteria species, e.g. Streptococcus pyogenes, Streptococcus mutans, Campylobacter jejuni, N. meningitidis, Escherichia coli, Francisella novicida, and other species known to contain type II CRIPSR system (Fonfara et al., 2013). The genes of such Cas9 enzyme, or other Cas enzymes, can be cloned from either the bacterial genomic DNA or cDNA using cloning techniques known in the art.

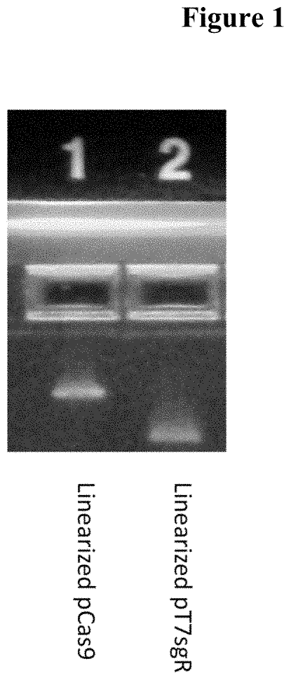

In another aspect, a cas9 gene is cloned behind a promoter, such as that of bacterial phage T7 RNA polymerase, T3 RNA polymerase, or Sp6 RNA polymerase, or other RNA polymerases. The cassette that encompass the promoter, the cas9 coding DNA, a fragment that encodes a poly(A) tail to mRNA suitable for the stability and expression in eukaryotic cells, can be used for in vitro translation (IVT) as a linear template or cloned into a vector such as a plasmid, a phagemid, or other carriers of DNA sequences (for example FIG. 1). One example of such vectors is the pIVT plasmid that the inventor described previously (Warren et al., 2012).

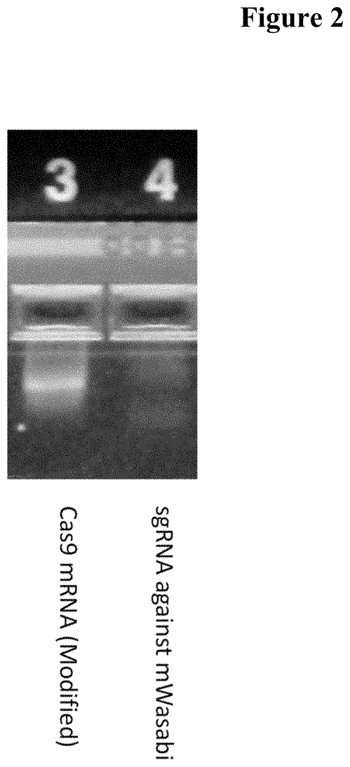

Disclosed herein are methods of generating mRNAs encoding Cas proteins. In one embodiment, mRNA is produced by in vitro transcription under optimized conditions as described herein. An embodiment of the disclosure are synthetic mRNA transcripts that serve as efficient templates for translation in living cells by incorporating a 5′ diguanosine cap and a poly(A) tail. The cap and tail can be incorporated into IVT transcripts enzymatically or co-transcriptionally. Benefits of enzymatic capping include high RNA yields, low costs, and a potential for producing almost pure capped RNA. However, as there is no easy way to check that enzymatic capping has proceeded successfully, it is preferred to use the more robust co-transcriptional capping approach. In this scheme a synthetic cap analog is included at high concentration in the IVT reaction buffer, the cap being preferentially incorporated in place of GTP at the 5′ end of transcripts based on the reagents' respective reaction concentrations. Another embodiment is to use a co-transcriptional approach to polyadenylate transcripts: a poly(dA:dT) tract at the end of the IVT template drives incorporation of the tail by the RNA polymerase. It is also an embodiment of the disclosure that the cas9 mRNA's poly(A) tail is added to the end of the coding region by a polyadenylation polymerase (FIG. 2).

In one aspect, in vitro transcription is preferably carried out with modified nucleotide triphosphates (NTPs), such as 5-methyl-Cytosine, 2-Thio-Uracil, or pseudouracil, or other modified nucleotides able to substitute unmodified nucleotide in RNA molecules that do not significantly alter the RNA's functions. Using modified nucleotides help reduce cellular immune response, which is particularly important when repeated deliveries of mRNA into the host cells are required to achieve desired level of genome modification among host cells, or the host cells are hypersensitive to exogenous RNA molecules.

The current disclosure further relates to generation of sgRNAs. Previously, sgRNAs as guide for CRISPR/CAS are introduced via a DNA vector or viral vector, whereby sgRNA-encoding DNA is placed behind a promoter that can drive transcription of short RNAs, e.g. U6 or H1 promoters. As an embodiment of the current invention, sgRNA encoding DNA is placed behind a promoter that is suitable for in vitro transcription, e.g. a T7, T3, or Sp6 promoter (FIG. 1). The cassette that encompasses the promoter and the sgRNA coding DNA can be used as a linear template or cloned into a vector such as a plasmid, a phagemid, or other carriers of DNA sequences. A transcription termination can also be achieved by having a transcription terminator sequence. One example of such a vector is the pIVT plasmid described previously (Warren et al., 2012). In one embodiment of the disclosure, sgRNAs are created by IVT using modified or unmodified NTP (FIG. 2).

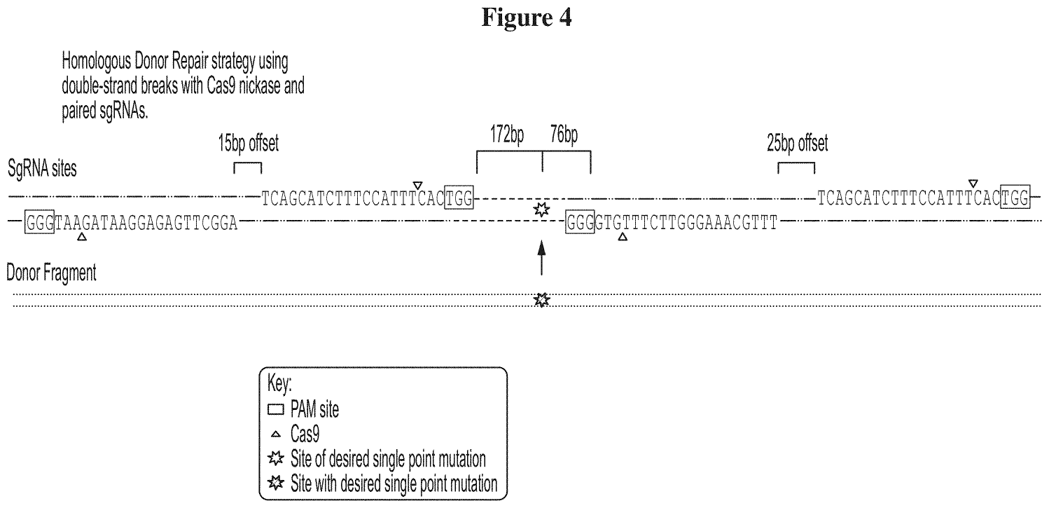

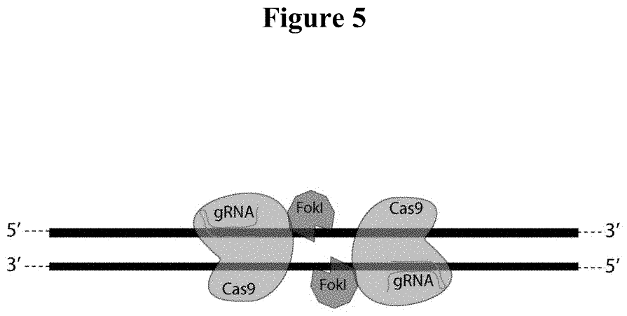

One aspect of the disclosure relates to the design of the Cas9 enzyme. The wildtype Cas9 enzyme naturally has two endonuclease functional domains SEQ ID NO: 1. By selective point mutation as described herein, the Cas9 enzyme can be converted from a dsDNA cutting enzyme into a single-strand DNA (ssDNA) nicking enzyme, e.g. SEQ ID NO: 2, SEQ ID NO: 3. Additionally, when two such nicking enzymes are on opposite strands of a dsDNA molecule, a double-stranded break can still be created, but as opposed to a double-stranded break created by a wildtype Cas9, two sgRNAs are needed, thereby providing added sequence-specificity to the process (FIG. 4). In one example, mRNA is created to express such mutants of Cas9 that nicks one strand when guided by one sgRNA. In another embodiment, the cas9 mRNA encodes a version of Cas9 further mutated to remove both of its endonuclease domains (SEQ ID NO: 4) and fused to an artificial nuclease domain such that of restriction enzyme FokI or other such restriction enzymes (FIG. 5). The resulting mutant form of Cas9 needs to form a dimer to function as an endonuclease, requiring the target sites defined by the pair of sgRNA sequences to be close together, preferably with a distance between about 5-30 or about 10-20 nucleotides (nts), or about between 12-18 nts, providing further specificity.

Another aspect of the current invention relates to the selection of CRISPR/CAS target sites. The design of a preferred sgRNA matching site on a eukaryotic genome is well established. In one embodiment of the current invention, in order to maximize target specificity during a chromosomal knock-in process (replacing a segment of sequence, which can be as short as a single nt, of the chromosome with another by providing a DNA template), it is hereby disclosed that two double-stranded cuts are made by using either nicking Cas9 mutants or a Cas9-FokI fusion when choosing the target sites. An example is illustrated in FIG. 4.

In one additional embodiment, the Cas9 or its nicking or blunt mutants is in-frame fused to epigenetically modifying enzymes, such as protein arginine methyltransferases PRMT1 and PRMT4 (CARM1), DNA methyltransferases, histone methyltransferases, histone acyltransferases etc. When introduced into target cells together with sgRNA, such fusion Cas9 enzymes will, instead of or in addition to cutting or replacing the dsDNA sequence, modify epigenetic information such as DNA methylation, histone acetylation, etc.

In one aspect of using RNA for providing sgRNA, the guide RNA, structure RNAs as in a typical sgRNAs, and if necessary, a linker RNA, can be further fused to a patch template RNA for local repair after cutting by Cas9 enzyme. It is known in the field that RNA can be used for homologous DNA break repair, which is incorporated herein by reference (Storici et al., 2007).

In another embodiment, a DNA or RNA aptamer that specifically binds to Cas9 is linked to a sequence replacement “patch” template in order to achieve gene knock-in or knock-out through the use of a template polynucleotide. By physically being attached to the Cas9 enzyme, the patch can be delivered close to the site of CRISPR/CAS cutting. The patch template can be either DNA or RNA.

One significant embodiment of the current disclosure relates to delivering the cas mRNA and sgRNA at appropriate absolute and relative doses. One of the unique advantages of using RNA as the form of delivery of genetic information is that it is more controllable in expression than using DNA. For expression of protein such as an enzyme, the mRNA molecules do not need to translocate into the nucleus, thereby eliminating a bottleneck typically presented by nuclear entrance, as well as many layers of uncertainty in terms of molar ratio between DNA and mRNA. The Cas proteins can be highly expressed immediately after the encoding mRNA enters cytoplasm by a transfection or electroporation process. Furthermore, it is also beneficial that the RNA molecules naturally have a relatively short half-life, therefore making the control of off-target effects of the CRISPR/CAS system more manageable than using DNA vectors or viral vectors.

A further embodiment of the current disclosure in relevance to dosing control relates to adjusting the ratio between gRNA Cas and mRNA. Because the dose of cas9 mRNA can be essentially proportionally correlated to the level of Cas enzyme, the all-RNA CRISPR/Cas system disclosed hereby enables a direct matching between the two component of CRISPR/Cas, namely the Cas enzyme and the gRNA, in order to obtain the highest on-target and the lowest off-target DNA cutting.

### EXAMPLES

### Example 1—Generation of Cas9 IVT Template

The DNA encoding Cas9 from bacterium Streptococcus pyogenes was codon maximized for optimal expression in mammalian, particularly human cells. The complete gene was assembled from 3 fragments generated through commercial gene synthesis service (Gene Oracle); mutations that disrupt DNA endonuclease domains were included during gene synthesis, resulting in different versions of cas9 as delineated in SEQ ID NOS: 1-4.

### Example 2—Production of Cas9 mRNA

Synthetic mRNA was generated in IVT reactions using a 4:1 ratio of anti-reverse cap analog (ARCA) to GTP to generate a high percentage of capped transcripts. Twenty percent substitution of 5m-CTP for CTP and 2-Thio-UTP for UTP in the nucleotide triphosphate (NTP) mix was employed to reduce the immunogenicity of the RNA products. ARCA and modified NTPs were purchased from Trilink Biotechnologies (San Diego). A 2.5×NTP mix was prepared (ARCA:ATP:GTP:C:5m-CTP:UTP:Pseudo-UTP at 15:15:3.75:3:0.75:3:0.75 mM). Each 20 μL IVT reaction comprised 8 μL NTP mix, 2 μL 10×T7 Buffer, 8 μL DNA template and 2 μL T7 enzyme (Promega). Reactions were incubated 4-6 hours at 37° C. and then treated with 1 μL RNAse-free DNase for an additional 30 minutes at 37° C. before being purified on a spin column, the RNA product being eluted in a volume of 80 μL. 8 μL 10×PAP buffer and 8 μL 10 mM ATP and 2 μL PAP (NEB) were added for 10 min to add poly(A) tail, followed by 3 μL Antarctic Phosphatase (New England Biolabs) for 10 min to remove immunogenic 5′ triphosphate moieties from uncapped transcripts and 10 μL of reaction buffer. Phosphatase reactions were incubated for 30 minutes at 37° C. and the IVT products were repurified if necessary (FIG. 2).

### Example 3—Production of sgRNA by IVT

Synthetic sgRNA was generated in IVT reactions using a 4:1 ratio of ARCA cap analog to GTP to generate a high percentage of capped transcripts. Twenty percent substitution of 5m-CTP for CTP and 2-Thio-UTP for UTP in the nucleotide triphosphate (NTP) mix was employed to reduce the immunogenicity of the RNA products. Cap analog and modified NTPs were purchased from Trilink Biotechnologies. A 2.5×NTP mix was prepared (ARCA:ATP:GTP:C:5m-CTP:UTP:Pseudo-UTP at 15:15:3.75:3:0.75:3:0.75 mM). Each 20 μL IVT reaction comprised 8 μL NTP mix, 2 μL 10×T7 Buffer, 8 μL DNA template and 2 μL T7 enzyme (Promega). Reactions were incubated 4-6 hours at 37° C. and then treated with 1 μL RNAse-free DNase for a further 30 minutes at 37° C. before being purified on a spin column, the RNA product being eluted in a volume of 80 μL. 3 μL Antarctic Phosphatase (New England Biolabs) was added for 10 min to remove immunogenic 5′ triphosphate moieties from uncapped transcripts and 10 μL of reaction buffer. Phosphatase reactions were incubated for 30 minutes at 37° C. and the IVT products were repurified if necessary (FIG. 2).

### Example 4—Modifying a Reporter Gene in Human Cells

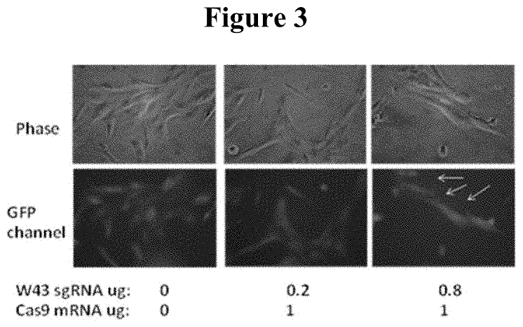

To demonstrate the utility of the disclosed system, a complete all-RNA CRISPR/CAS system was created to disrupt a fluorescent protein (FP) mWasabi (Allele Biotech) permanently expressed in mammalian cell NIH-3T3. NIH3T3-mWasabi cells were grown at 15% confluency in serum-free medium, cas9 mRNA and sgRNA were co-transfected into the cells; after 2 hrs serum-containing medium was added. As illustrated in FIG. 3, from left to right, cells received 0, 0.2, or 0.8 ng of sgRNA against the mWasabi site nt43 (W43), as indicated below each panel. The top panels show where the cells are (phase contrast); bottom panels show the cells that are still fluorescent (green fluorescence channel). The three arrows in the right-bottom panel point to the cells that lost the green fluorescence in the well that received the higher dose of sgRNA together with cas9 mRNA. No cells in the 0 or 0.2 ng sgRNA wells lost the green fluorescence.

### Example 6—Method Embodiments for Generating a Single Base Pair Mutation Via an mRNA-Based CRISPR/Cas9 System

### A. Sequence Design:

I. Exemplary method embodiments for sequence design of sgRNA:

1) A 300 bp sequence surrounding the intended mutation site is run through the web-based sgRNA design tool. (“MIT Crispr Design Tool” MIT). 2) Guide RNA selection is determined by 2 parameters: a) proximity to intended mutation, and b) potential off target score. 3) A minimum of two sgRNA sites are selected. (Optimal parameters would be a PAM site within 5 bp of intended mutation and a sgRNA score of >70.)

II. Exemplary method embodiments for design of single stranded oligonucleotide donor (ssODN) repair template:

1) Obtain a 60-100 bp sequence with homology arms centered on intended mutation. 2) Optionally: engineering a silent mutation to destroy protospacer adjacent motif (PAM) site (i.e. from NGG to NGT, NGA, or NGC). 3) Optionally: engineering a silent mutation <10 bp away from intended mutation to create a restriction site. This can facilitate the screening process. 4) Obtain ssODN via IDT “Ultramer” service (standard desalted 4 nmoles) (Integrated DNA Technologies, Coralville, Iowa).

III. Exemplary method embodiments for genomic DNA amplification:

1) BLAST searching the genomic region for pseudo genes or other highly similar genomic sequences. 2) Designing and testing multiple sets of primer pairs to amplify a ˜400-600 bp region centered around the intended mutation using genomic DNA lysate template. 3) Choosing the best primer pair for screening CRISPR treated cells based on robustness of amplification (i.e. high yield and no non-specific bands). 4) Sequencing the PCR product to verify quality sequencing reads of amplicon.

IV. Exemplary primers for qPCR based screening:

1) Selecting Tm for qPCR primers to be ˜64° C. 2) The forward primer can be ˜100 bp away from intended mutation, and contained within the amplicon generated from step III. 3) The reverse Primer (mutation specific) can have the intended mutation at the 5′ leading end.

B. Exemplary Method Embodiments for In Vitro Transcription (IVT) of sgRNA and Cas9 Wt mRNA.

I. For IVT template production of sgRNA. 1) design and synthesize a forward primer with the following 3 elements: a) a T7 promoter, b) the protospacer element sequence (step A. I.2), and c) a crRNA specific sequence. A universal reverse primer (sgRNA_Rev) is used to complete the primer pair. 2), using these primers and the pT7sgRNA plasmid as a template, a PCR reaction is performed to create the IVT template (˜131 bp). DpnI digesting the reaction sample and perform a PCR cleanup, so it can be suitable for the in vitro transcription reaction.

II. Exemplary method embodiments for IVT template production of Cas9 wt. 1) using the pIVT-Cas9 wt plasmid as a template and the INS-F+d(T)120-Rev (SEQ ID NO: 9) as the primer pair, a PCR reaction is performed to create the IVT template. 2) Perform a PCR clean-up on resulting PCR product so that it is suitable for in vitro transcription.

III. Exemplary method embodiments for IVT reaction to produce CRISPR elements. 1) using the templates created via PCR, perform an IVT reaction to transcribe the sgRNA and Cas9 wt mRNA. 2) Purify and QC transcripts via gel imaging and Bioanalyzer (Agilent).

IV. Exemplary method embodiments for validation of IVT sgRNA via in vitro cleavage test. 1) Create a cleavage template for validating IVT transcribed sgRNA by amplifying a fragment of genomic DNA containing the target sequence. (Made in step A. III.3). 2) Performing a cleavage reaction of the cleavage template using sgRNA from B. III.1 in combination with recombinant Cas9 nuclease (see Protocol III below). 3) Complex Cas9 and sgRNA at a 1:1.2 ratio respectively. 4) Incubate RNP complex with cleavage template amplicon at a 10:1 ratio, then run reaction on agarose gel. 5) Analyze gel to assess cleavage efficiency by observing lower molecular weight cleavage bands.

C. Exemplary Method Embodiments for qPCR SYBR Green Based Screening.

I. Construction of plasmid and amplicon standards: 1) Via Gibson assembly, sub-cloning region of interest (amplified from genomic DNA template using primers with pIVT compatible overlaps) into the pIVT vector. The Insert is between 400-600 bp. This is designated as the “WT” vector. 2) Using the QuickChange site directed mutagenesis kit (Agilent), generating the intended single point mutation (follow kit procedure to design mutagenesis primers and for thermal cycling parameters). The resulting construct is designated as the “Mutant” vector. 3) Amplifing “WT” and “Mutant” constructs with primers designed in A. III to create “WT” and “Mutant” amplicons. Optional: Sanger sequence purified PCR products to confirm WT and Mutant sequences. 4) Quantifying the amplicons using a Nanodrop spectrophotometer, then standardizing the concentration by diluting amplicons down to 60 fg/μl each. After standardization of concentration is done, assembling the following ratios:

0% “Mutant”, 100% “WT”

1% “Mutant”, 99% “WT”

10% “Mutant, 90% “WT”

50% “Mutant”, 50% “WT”

II. Exemplary method embodiments for Assaying standards on qPCR: 1) Set up qPCR plate with: a) Template: standards (include duplicates) created in step I. b) Primers: Using primers designed in A. IV. 2) Run a Standard Quantification RT-PCR program with SYBR green reporter and compare Ct values of each standard point. Ct values are reflective of the relative mutant population ratio (higher mutant ratios yield lower Ct values). 3) The 1% “Mutant” standard has about ΔCt of ≥2 when compared to 100% “WT.” With a ΔCt of ≥2, the qPCR-based screening method can reliably detect mutations with a sensitivity of at least 1%.

D. Exemplary Method Embodiments for Transfection of Target Cells (iPSC).

I. Plating of exemplary target cells: 1) Cells are cultured in E8 media supplemented with ROCK Inhibitor (Y27632) during passages. 2) The day before transfection, cells are passaged into a 6-well plate at a density of 2.5×10⁵ cells/well.

II. Exemplary method for Transfection of CRISPR elements: 1) The day after seeding, the cell density is least double and exhibit small clusters of one to four cells. 2) Transfecting cells with IVT RNA CRISPR elements produced in B. III.1 and ssODN ordered in A. II.4. using Messenger Max transfection reagent. In addition, performing a negative control transfection containing only the ssODN. To gauge the transfection efficiency, the negative control should also contain 100 ng mRNA encoding a fluorescent protein such as mNG. 3) Replacing transfection media with fresh pre-warmed E8 media (supplemented with Y27632) four hours after transfection. 4) Next day (˜12-18 hrs later), mNG expression in the negative control well is checked. When expression is robust, proceed with a second transfection of sgRNA and ssODN in the experimental and negative control wells. Cas9 mRNA can be delivered repeatedly in the repeat transfections. Four hours after the repeat transfection, replace transfection media with fresh E8 (with Y27632). 5) Culturing cells for 2 more days, then passage at a 1:3 dilution into another 6-well plate. Leftover cells are lysed and analyzed.

### E. Exemplary Method Embodiments for Screening and Cloning of CRISPR Treated Cells.

I. Exemplary method embodiments for lysis of treated cells and amplification of gDNA for screening. 1) Performing lysis of leftover cells from D. II.5 experimental and negative control wells. Resuspending cells in Allele's Mouse Tail lysis buffer (Allele Biotech, San Diego) and run samples using lysis program in thermocycler. The resulting lysate is amplified (<26 cycles) using primers designed in A. III.3 using Herculase II fusion DNA polymerase (Agilent Technologies). Performing PCR cleanup on the PCR product. The resulting experimental and negative control amplicon libraries is designated as the experimental and negative control “Bulk populations.” 2) Using the Nanodrop to quantify the PCR product, performing a dilution to standardize all amplicons to 60 fg/μl.

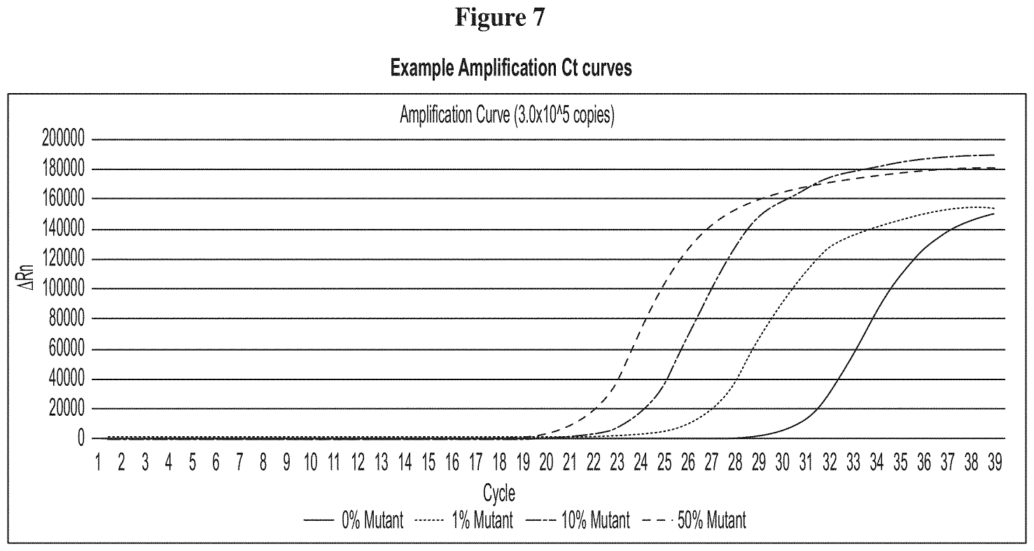

II. Exemplary method embodiments for screening bulk populations: 1) Performing SYBR green based Standard Quantification qPCR screening, with the Bulk amplicon libraries made in previous step and the standards made in C. I.4. 2. When the ΔCt between experimental and negative control libraries is ≥2, and within the range of 1% mutant population according to standards, proceed to the single cell-cloning step. See FIG. 7

III. Exemplary method embodiments for single cell, 96-well plate passaging: 1) Disassociating cells from passage 2 CRISPR experimental cells using TrypLE. Pass cells through a 70 μm cell strainer to produce a single cell suspension, then determine cell count and calculate a dilution that will produce 2-3 cells/100 μl; 2) In pre-warmed E8 (supplemented with Y27632), seed 2-3 cells/well (100 μl/well) in four Matrigel-coated 96-well plates; 3) Next day, quickly confirm by microscope presence of attached cells. Wells should have between 0-3 cells per well (it is unnecessary to inspect every well). Perform half media changes daily (aspirate 50 μl, add 50 μl pre-warmed E8 supplemented with Y27632); 4) After growth into ˜50-100 cell cluster has been established (typically about seven days), switch to non Y27632 supplemented E8 and media changes every other day.

IV. Exemplary Method Embodiments for Making Duplicate Plates for Screening:

1) Once cells have reached >70% confluence, passage ¼^{th} of cells onto a new Matrigel coated 96-well plate with EDTA into Y27632 supplemented E8 media. The resulting plate is designated as the “duplicate plate”. Leaving the remaining ¾th of cells in the source plate with fresh pre-warmed Y27632 supplemented E8 media (cells will reattach). 2) Replacing media daily with Y27632 supplemented E8 for both duplicate and source plates. After 3-5 days the source plate should be ready for lysis and analysis.

V. Exemplary method embodiments for screening of clonal plates: 1) Performing lysis protocol (same as E. I.1) on source plate. Performing a test PCR on 3 wells with 2 lysate volumes to identify optimal lysate template volume. 2) Performing plate PCR of cell lysate from source plate. Once PCR is complete, run the PCR products from the plate on a large format agarose gel to confirm amplification and provide record for any variation in amplification yield. 3) Using the SurfaceBind Purification Plate (Allele Biotech), purifying PCR product according to protocol. Elute in 30 μl Elution Buffer. 4) Performing a 1:1000 dilution with the purified PCR product into molecular grade water. Use 2 ml collection plate to maintain plate format. The amplicon library is now at a suitable concentration for screening. 5) Performing SYBR green based Standard Quantification qPCR screening on amplicon libraries from the four 96-well plates. Optionally: include a positive control (1% mutant standard library) and a negative control (using negative control amplicon library) in any well positions corresponding to empty wells in the plates (i.e. where cells failed to attach/grow). 6) The most left-shifted qPCR Ct curves (the “Outliers”) represent wells having the highest probability of containing a mutant cell population (i.e. with the intended HDR event). Performing Sanger sequencing analysis on the original purified amplicon library stock corresponding with all outlier wells.

VI. Exemplary method embodiments for selection of clones and expansion: 1) Analyzing sequencing results from E.V.6 to confirm presence of intended mutation, and to determine the relative size of the mutant population (i.e. in the case of a mixed population) based on the ratio of peaks in the chromatogram. Expand confirmed outlier wells by passaging from the duplicate plate made in E. IV.1. In the first round of expansion, passage a single well from the 96-well plate to a single well in a 12-well plate.

a) When sequencing results indicate a mixed population, a second round of single-cell cloning is performed (repeat steps starting from E. III). After cells reach confluency in the 12-well plate (˜10⁵ cells), expand and freeze the confirmed outliers, and proceed to a second round of single-cell cloning. Recommended: lyse, amplify and sequence any remaining cells to test whether the mutation is preserved after passaging.

b) When sequencing results indicate a pure population (i.e. the ratio of chromatogram peaks corresponding to WT and Mutant are 1:1 [indicating a heterozygous population]), confirm the cells are heterozygous by performing a second analytical round of single-cell cloning by analyzing 24 to 48 wells. Expanding cells to a 6-well plate format. Cryopreserve cells at a concentration of 10⁶ cells/vial. Lyse, amplifying and sequencing a portion of the cells to test when mutation is preserved after passaging.

### Protocols

I. Exemplary method embodiments for Production of sgRNA IVT template.

Materials: -pT7sgRNA plasmid; sgRNA Rev Primer; Custom sgRNA forward primer; 10 mM dNTPs; Phusion Polymerase (New England Biolabs) with 5×GC buffer; DpnI restriction enzyme; NucleoSpin® Gel (Clontech) and PCR Clean-up; Molecular Grade H₂O; 1% agarose gel/1×TAE running buffer; Bioline 1 kb DNA Ladder

Assemble PCR reaction as follows in Table 1:

TABLE 1
Assembly of PCR Reaction

| | |
|---|---|
| 1 μl | pT7sgRNA Template (~50 ng) |
| 2 μl | Custom sgRNA forward primer (10 uM) |
| 2 μl | sgRNA Rev primer (10 uM) |
| 8 μl | 5x PCR buffer (GC) |
| 0.8 μl | 10 mM dNTPs |
| 0.4 μl | Polymerase (NEB Phusion) |
| 40 μl | TOTAL VOLUME |

After assembly, run program as depicted in Table 2

TABLE 2
Run Program

| | | |
|---|---|---|
| 95° C. | 3 | min |
| 95° C. | 30 | s |
| 67° C. | 30 | s |
| 72° C. | 20 | s |
| 30 cycles | | |
| 72° C. | 5 | min |

| | |
|---|---|
| 10° C. | Hold |

2) Run 2 μl of PCR product on a 1% Agarose gel with a 1 Kb DNA Ladder to confirm yield and correct size of 131 bp.

3) Add 1 μl of DpnI enzyme directly to PCR reaction and incubate at 37° C. for 15 min to digest the template plasmid.

4) Perform PCR cleanup using NucleoSpin kit according to manufacturer's protocol.

5) Template is ready for in vitro transcription reaction.

II. Exemplary method embodiments for IVT template production of Cas9WT.

Materials:
pIVT-Cas9WT plasmid
Tail 120 Reverse Primer
Insert-F forward primer
KAPA Biosystems' HiFi HotStart ReadyMix
NucleoSpin® Gel and PCR Clean-up
Molecular Grade H₂O
1% agarose gel/1×TAE running buffer
Bioline 1 kb DNA Ladder
1) Assemble PCR reaction as follows Table 3:

TABLE 3
PCR Reaction Components

| | |
|---|---|
| 1 μl | pIVT-Cas9wt Template (~10 ng) |
| 12 μl | Tail 120 Reverse (1 μM) |
| 12 μl | Insert-F (1 μM) |
| 25 μl | Kapa HiFi ReadyMix |
| 50 μl | TOTAL VOLUME |

2) Run 2 μl of PCR product on a 1% Agarose gel with a 1 Kb DNA Ladder to confirm yield and correct size of ˜4.5 kb.

3) Perform PCR cleanup using NucleoSpin kit according to manufacturer's protocol.

4) Template is ready for in vitro transcription reaction.

III. Exemplary method embodiments for Expression of recombinant Cas9.

Materials:
pCold-Cas9Wt plasmid
SOC media
2XYT media
Carbenicillin
LB-agar plates
NEB Express competent Cells
1M IPTG
High-density cobalt resin
Coupling Buffer (100 mM Phosphate, 150 mM NaCl)
Lysis Buffer (50 mM NaPO₄, 300 mM NaCl, 5 mM Imidazole)
Elution Buffer (100 mM NaPO₄, 150 mM NaCl, 200 mM Imidazole)
Dialysis Buffer (300 mM NaCl, 10 mM Tris-HCl pH 8.0, 0.1% Tween

a) Bacterial expression:

1) Transform the E. coli host strain (NEB Express) with pCold-Cas9Wt plasmid and select the transformants on a LB-Carbenicillin selection plate.

2) Inoculate the transformant in 5 ml medium including (100 μg/ml of Carbenicillin), and culture at 37° C. with shaking for 24 hours.

3) Next day, add the growing 5 ml culture into a large 2.5 L flask with 500 ml 2XYT-Carb. At OD600=0.4-0.5, cool the culture solution to 15° C. quickly and let stand for 30 minutes.

4) Add IPTG at a final concentration of 0.1-1.0 mM, and continue the culture with shaking at 15° C. for 24 hours.

5) Remove overnight cultures from shaking incubator.

6) Pour culture into clean Oakridge tubes.

7) For cultures of 500 mL or greater, only able to pour half the culture into the tubes.

8) Spin the Oakridge tubes at room temperature for 10-15 minutes at 5,000 g in the Sorvall centrifuge.

9) Ensure that the seams of the Oakridge tubes are not facing the center of the rotor to avoid breaking the tubes.

10) Decant the supernatant from the tubes when the spin is complete.

11) Repeat the prior 3 steps when working with a large culture.

B. Cell Lysis

1) Resuspend the pellet present in the Oakridge tubes by adding 25 mL of Lysis Buffer and gently swirling.

2) Once the pellets have been completely resuspended, pour the resuspension into 50 mL ultra high performance tubes.

3) Bring the volume of the 50 mL tubes up to 50 mL using Lysis Buffer.

4) Split this 50 mL volume up into two 50 mL ultra-high performance tubes. (25 mL in each).

5) Place both tubes into the freezer (−20) until completely frozen (or for long-term storage). A completely freeze normally takes 1-3 hours.

6) Remove a tube from the freezer and thaw completely.

7) Add several drops of De-foaming agent (2-3).

8) Place the tube on ice and sonicate for 3 minutes at maximum.

*Be careful that the probe of the sonicator does not touch the bottom of the tube, but is rather close to it.

9) Place tube into the Eppendorf centrifuge and spin for 15 minutes at 4° C. and maximum speed.

10) Make sure that the centrifuge is balanced properly.

11) While tubes are spinning down, pour about 5 mL of cobalt slurry into a sterilized 50 mL tube.

12) Add 20 mL of Lysis Buffer to the cobalt slurry.

13) When the cobalt resin settles to the bottom, pour off the lysis buffer.

13) Once spin is complete, filter the lysate (the supernatant) using a 0.7 um syringe filter and add it to the cobalt resin.

14) Tumble at 4° C. for 10-30 minutes.

c. His-Tag Purification

1) Pour protein/cobalt slurry over a drip column and allow it to drain completely. It is not necessary to save the flow-through or any subsequent washes.

2) Wash the 50 ml tube that previously contained the protein with 15 ml of lysis buffer.

3) Pour this wash over the drip column.

4) Wash column with 10-15 mL of Coupling Buffer. Allow it to drip through.

5) Place 15 ml sterile collection tubes underneath the column(s).

6) Pour 15 ml of Elution Buffer over the column and collect the eluted protein in said tube.

7) Measure the concentration of the protein and store at 4° C. until needed.

d. Dialysis

1) Filter protein through a 0.45 μm syringe filter into a 30 kD spin column filter unit. Add dialysis buffer (as needed) to the filter unit to bring the total volume to 15 mL.

2) Place filter unit into the centrifuge (swinging bucket rotor) and spin at RT for 20 minutes at 4000 g, or until the volume remaining in the filter unit is 1 mL or less.

3) Remove filter unit from centrifuge. Discard the flow-through. Add appropriate amount of dialysis buffer to bring the total volume back up to 15 mL. Invert the filter unit to mix.

4) Repeat until a dilution factor of at least 4,000 has been achieved. The dilution factor can be calculated as follows: df=(Vfinal/Vinitial).

IV. Exemplary method embodiments for in vitro transcription of sgRNA and Cas9WT.

Materials:
Anti Reverse Cap Analog, ARCA
2-Thio-UTP
5-Methy-CTP
rATP
rUTP
rGTP
rCTP
T7 RNA Polymerase
Transcription optimized 5×Buffer
DTT 100 mM
1M MgCl₂ Solution
RQ1 RNase-free DNase
Antarctic Phosphatase
10× Antarctic Phosphatase Reaction Buffer
TE buffer pH=8.0
RNA Clean & Concentrator™-25
TE buffer pH=7.0

1) Assemble IVT reaction as follows in Table 4:

TABLE 4
Components of IVT Reaction

| Reagent | Volume (μl) |
|---|---|
| NTP | 16 |
| DTT, 100 mM | 4 |
| Transcription | 8 |
| Optimized 5X buffer | |
| MgCl₂, 1M | 0.34 |
| T7 RNA Polymerase | 4 |
| (20U/μl) | |
| Template DNA (made | 8 (~500-800 ng) |
| in Exemplary | |
| Protocol I and II | |
| above | |

2) Note:
Before adding the Template DNA to the 1.5 ml sterile microcentrifuge tube, add the Template DNA to a PCR tube. Place this PCR tube in the PTC-100 Programmable_thermal controller that has been pre-heated to 37° C. With P200 pipette, transfer the 32 μl ready-to-use master mix to each reaction. Pipette up and down 5 times in order to mix well.

3) Incubate this mixture for 4-6 hours at 37° C. in the T100 Thermal Cycler.

4) After performing the in vitro transcription reaction as completed in Step 8.2.3, add 2 μl of RQ1 RNase-free DNase to each reaction in order to remove the DNA template.

5) Incubate the mixture for at least 30 minutes at 37° C. in the T100 Thermal Cycler. After the incubation period from Step 5.2.5 is complete, add 5 μl of 10× Antarctic phosphatase reaction buffer and 3 μl of Antarctic Phosphatase to each reaction.

6) Incubate the mixture for at least 30 minutes at 37° C. in the Thermal Cycler.

7) After the incubation in Step 7 is complete, check the mRNA on an E-gel.
i) For each sample, add 9 μl of TE buffer pH=8.0 with 1 μl of the prepared mRNA in separate PCR tubes using a P20 pipette and appropriately sized tip. With the same tip, gently swirl the tube contents to mix.
ii) Proceed to load each 10 μl mixture to each well on the E-gel. Each sample occupies one well.
iii) Run the built-in program of the E-gel electrophoresis system for 8 minutes. Refer to the E-gel iBase Power System Equipment Manual for operating instructions.
iv) Check the RNA bands with LED light. Determine when a clear RNA band has appeared at the correct size position Cas9 size: ˜4400 nt; sgRNA size: ˜150 nt
v) When a single, clear RNA band at the correct size position is observed, proceed to Step 8.

8) Purify the mRNA with RNA Clean & Concentrator™-25 according to manufacturer's protocol.

9) Quantify RNA products on the Nanodrop. Cas9WT mRNA and sgRNA are now ready for downstream use.

V. Exemplary method embodiments for iPSC culture.

Materials
TeSR™-E8™
Corning® Matrigel®
Tissue culture-treated cultureware
DPBS
Y-27632 (ROCK inhibitor)
PRG-1 EDTA
TrypLE 1×
Costar™ Sterile Disposable Reagent Reservoirs
Tissue culture grade 96 well plates
Mr. Frosty (Thremo Scientific)
DMSO
HSA
Opti-MEM
MessengerMax Transfection Reagent (Thermo Fisher Scientific)

a.) Thawing iPSCs
1) At least one hour prior to thawing, coat 1 well of a 6-well plate with Corning@ Matrigel® (1 mL per well using 1:80 dilution in DMEM);
2.) Prewarm 2 ml TeSR™-E8™ with 10 μM Y27632 in 5% CO₂ 5% O₂ cell culture incubator for 30 min.
3.) Take out one vial of iPS cell line from where it is stored in the LN tank or −80° C.
4.) Thaw the vial of cells immediately in 37° C. waterbath;
5.) Rinse the vial completely with 70% ethanol, put the vial in cell culture hood;
6.) Add the cells dropwise to 10 ml Dulbeccos's phosphate-buffered saline (DBPS) with calcium and magnesium in 15 ml tube;
7.) Centrifuge at room temperature at 200 g for 2 mins;
8.) Rinse the tube completely with 70% ethanol, put the vial in cell culture hood;
9.) Remove the supernatant, add the pre-warmed 2 ml E8 medium with 10 uM Y27632, Gently pipette up and down to resuspend cells.
10.) Add the 2 ml cell suspension into a single well of the Matrigel-coated plate, tap the plate to mix the cells gently.
11.) Label the plate with the NAME of the cell lines and the Passage. Put the flask into a 37° C. 5% CO₂ 5% O₂ cell culture incubator;
12.) Change the medium every other day (supplement media with 10 uM Y27632 until colony size exceeds 50-100 cells).

b.) Passaging (6-well plate)
1.) At least one hour prior to passaging, coat tissue culture treated plates with Corning® Matrigel® (1 mL per well using 1:80 dilution in DMEM).
2.) Aliquot sufficient TeSR™-E8™ (StemCell Technologies) (2 mL per well in 6-well plate) and warm to room temperature (15-25° C.).
3.) Wash cells with 1 mL of phosphate-buffered saline (PBS) without Ca²⁺ and Mg²⁺ and aspirate. Note: There is no need to remove regions of differentiated cells.
4.) Add 0.3 mL of PRG-1, then aspirate most of the PRG-1 within 15 s leaving ˜80 uL in the well (so that colonies are exposed to a thin film of liquid).
5.) Incubate at 37° C. for 3-5 minutes.
6.) Tap plate gently to aid detachment. Add 1 mL of TeSR™-E8™.
7.) Detach the colonies by light pipetting. Take 50-250 μl of cell/media mixture and seed into the new Matrigel coated 6-well plate. Add 2 ml of Y27632 supplemented TeSR™-E8™ to seeded wells.
8.) Place plate into a 37° C. 5% CO² 5% 0² cell culture incubator. Change media every other day (supplement media with 10 μM Y27632 until colony size exceeds 50-100 cells).

c.) Passaging Single Cell (96-well plate)
1.) At least one hour before passaging, coat new 96 plates with Corning® Matrigel® (50 μl/well using 1:80 dilution in DMEM).
2.) Aliquot sufficient TeSR™-E8™ and warm to room temperature (15-25° C.). Approximately 12 ml of TesR-E8 is needed for each 96-well plate.
3.) Wash cells with 1 mL of phosphate-buffered saline (PBS) without Ca²⁺ and Mg²⁺ and aspirate.
4.) Add 0.4 mL of TrypLE (to dissociate to single-cell) and aspirate within 15 s, so that colonies are exposed to a thin film of liquid.
5.) Incubate at 37° C. for 3-5 minutes.
6.) Tap plate to aid detachment. Add 2 mL of Y27632 supplemented TeSR™-E8™ and pipette up and down. Pipette up the cells and strain them using a 37 μm cell strainer into a 15 ml conical tube.
7.) Perform a cell count using Moxi Z cell counter and Moxi Z cassette by pipetting 75 μL of cells from step 6 into the fill port of the cassette. The read out will be in cells/mL
8.) In most cases, cell counts should be between 300,000 to 500,000 cells/ml. Carry out serial dilution to get a 2-3 cells/100 μL concentration in Y27632 supplemented TeSR™-E8™.
9.) After 24 hours, check wells for single cells.
10.) Perform half media changes daily by removing 50 μl of media and adding 50 μl of fresh Y27632 supplemented TeSR™-E8™ until a ˜50-100 cell colony forms (usually 7 days). Proceed with full media changes (without Y27632) every other day until 80% confluency. Plate is now ready for duplication.

d.) Duplicating Plate (96-Well Plate)
1.) At least one hour before passaging, coat new 96 plates with Corning® Matrigel® (50 μl/well using 1:80 dilution in DMEM).
2.) Aliquot sufficient TeSR™-E8™ and warm to room temperature (15-25° C.). 20 ml of media is needed for each 96 well plate duplication.
3.) Wash cells with phosphate-buffered saline (PBS) without Ca²⁺ and Mg²⁺ (100 μl per well) and aspirate.
4.) Add 50 μl of PRG-1 EDTA to each well and aspirate 40 μL, so that colonies are exposed to a thin film of liquid.
5.) Incubate at 37° C. for 3-5 minutes.
6.) During incubation, add 75 μl Y27632 supplemented TeSR™-E8™ to each well of the duplicate 96-well plate prepared in step 1.
7.) Tap plate to aid detachment. Add 125 μl of Y27632 supplemented TeSR™-E8™ and pipette up and down.
8.) Pipette 25 μl of the 125 μl of detached cells into the duplicate 96-well plate. Make sure orientation of the plate is conserved. Source and duplicate plates should now both have 100 μl of media.
9.) Place plates in low oxygen incubator. Full media changes should be performed every other day until source plate is ready to be lysed and analyzed.

e.) Well/Clone Expansion
1.) At least one hour before passaging, coat a new 12-well plate with Corning@ Matrigel® (0.5 ml/well using 1:80 dilution in DMEM).
2.) Aliquot sufficient TeSR™-E8™ and warm to room temperature (15-25° C.).
3.) Wash selected wells with phosphate-buffered saline (PBS) without Ca²⁺ and Mg²⁺ (100 μl per well) and aspirate.
4.) Add 50 μl of PRG-1 EDTA to each well and aspirate 40 μL, so that colonies are exposed to a thin film of liquid.
5.) Incubate at 37° C. for 3-5 minutes
6.) Tap plate to aid detachment. Add 100 μl of Y27632 supplemented TeSR™-E8™ and pipette up and down.
7.) Pipette all 100 μl of cell media mixture into the 12-well plate prepared in step 1. Add an additional 1 ml of Y27632 supplemented TeSR™-E8™. Label wells with the appropriate source.
8.) Perform full media changes every other day until 80% confluency.
9.) Split cells onto a 6-well plate according to protocol outlined in V.b. These cells can proceed to cryopreservation step upon confluency.

f.) Cryopreservation
1.) Aliquot sufficient TeSR™-E8™ and warm to room temperature (15-25° C.).
2.) Wash cells with 1 mL of phosphate-buffered saline (PBS) without Ca²⁺ and Mg²⁺ and aspirate.
3.) Add 0.3 mL of PRG-1, then aspirate most of the PRG-1 within 15 s leaving ˜80 uL in the well (so that colonies are exposed to a thin film of liquid).
4.) Incubate at 37° C. for 3-5 minutes.
5.) Tap plate gently to aid detachment. Add 3 mL of phosphate-buffered saline (PBS) with Ca²⁺ and Mg²⁺.
6.) Detach the colonies by light pipetting. Transfer cells to a 15 ml conical tube.
7.) Centrifuge at 300×g for 3 minutes at room temperature to pellet cells. Aspirate PBS.
8.) Resuspend pellet in cryopreservation media (Y27632 supplemented TeSR™-E8™, 10% HSA, and 10% DMSO) so that the concentration is 1-0.5×10⁶ cells/ml.
9.) Transfer 1 mL of cell aggregates to a labeled cryovial.
10.) Freeze cell aggregates using Mr. Frosty in −80° C. freezer, followed by long-term storage at −135° C. (liquid nitrogen) or colder. Short-term storage (<3 months) at −80° C. is suitable.

g.) Passaging for Transfection
1.) A day prior to transfection seed 250,000 cells/well onto a Matrigel coated 96-well plate according to the following protocol:
   i. At least one hour before passaging, coat new 6-well plates with Corning® Matrigel® (1 ml/well using 1:80 dilution in DMEM).
   ii. Aliquot sufficient TeSR™-E8™ and warm to room temperature (15-25° C.).
   iii. Wash cells with 1 mL of phosphate-buffered saline (PBS) without Ca²⁺ and Mg²⁺ and aspirate.
   iv. Add 0.4 mL of TrypLE (to dissociate to single-cell) and aspirate within 15 s, so that colonies are exposed to a thin film of liquid.
   v. Incubate at 37° C. for 3-5 minutes.
   vi. Tap plate to aid detachment. Add 2 mL of Y27632 supplemented TeSR™-E8™ and gently pipette up and down. Strain cells using a 37 μm cell strainer into a 15 ml conical tube.
   vii. Perform a cell count using Moxi Z cell counter and Moxi Z cassette.
   viii. With the known cell count, add appropriate volume of cells such that 250,000 cells are seeded per well. Add appropriate amount of Y27632 supplemented TeSR™-E8™ to bring well volume up to 2 ml.
2.) After 12-18 hours, cells should be in small 2-5 cell clusters. Cell density should be around 70-80% prior to transfection.

h.) Transfection
1.) Equilibrate MessengerMAX transfection reagent and 5 ml of Opti-MEM at room temperature for 10 min.
2.) Assemble transfection complexes according to Table 5:

TABLE 5
Assembly of Transfection Complexes

| | | Opti- |
|---|---|---|
| | MessengerMAX | MEM |
| Tube 1-AM | 5 μl | 125 μl |
| Tube 2-AM | 1 μl | 50 μl |
| Tube 1-BM | 1 μl | 25 μl |
| Tube 2-BM | 1 μl | 25 μl |

3.) Incubate diluted MessengerMax for 10 min before mixing with diluted mRNA according to Table 6:

TABLE 6

| | | | mNG | Opti- |
|---|---|---|---|---|
| | Cas9 mRNA | sgRNA | mRNA | MEM |
| Tube 1-A | 1.5 ug | 0.35 ug | | 125 μl |
| Tube 2-A | | | 0.20 ug | 50 μl |

Dilute ssODN according to Table 7.

TABLE 7

| | ssODN | Opti-MEM |
|---|---|---|
| Tube 1-B | 1 μl at 10 μM | 25 μl |
| Tube 2-B | 1 μl at 10 μM | 25 μl |

Mix diluted mRNA and MessengerMax Transfection reagent as follows in Table 8:

TABLE 8

| Mix | Content |
|---|---|
| Tube 1-A with Tube-1AM | CRISPR elements |
| Tube 2-A with Tube-2AM | FP negative control |
| Tube 1-B with Tube-1BM | ssODN |
| Tube 2-B with Tube-2BM | ssODN |

Incubate complexes for 5 min.

4.) Remove media from the two wells to be transfected, and wash cells with 1 mL of phosphate-buffered saline (PBS) without Ca²⁺ and Mg²⁺ and aspirate.

5.) Add the transfection complex mixtures to the respective wells. Plate Setup below Table 9:

TABLE 9

| | |
|---|---|
| Well 1 | CRISPR elements and ssODN |
| Well 2 (negative control) | FP negative control and ssODN |

6.) Add Y27632 supplemented TeSR™-E8™ to each well so final volume is 600 μl. Place plate into low oxygen incubator.

7.) After 4-6 hours, aspirate transfection media, and replace with 2 ml Y27632 supplemented TeSR™-E8™. Allow cells to incubate overnight

8.) After 12-18 hours (or next morning) confirm transfection was successful by examining mNG fluorescence, then proceed to second transfection (sgRNA and ssODN only).

9.) Second round transfection: prepare transfection complex according to Tables 10-13 below:

TABLE 10
Dilute MessengerMAX:

| | MessengerMAX | Opti-MEM |
|---|---|---|
| Tube 1-AM | 2 μl | 100 μl |
| Tube 1-BM | 1 μl | 25 μl |
| Tube 2-BM | 1 μl | 25 μl |

Incubate diluted MessengerMax for 10 min before mixing with Cas 9 mRNA

TABLE 11
dilute sgRNA:

| | Cas9 | | mNG | Opti- |
|---|---|---|---|---|
| | mRNA | sgRNA | mRNA | MEM |
| Tube 1-A | 0 ug | 0.35 ug | | 100 μl |
| Tube 2-A | | | | |

TABLE 12
Dilute ssODN:

| | ssODN | Opti-MEM |
|---|---|---|
| Tube 1-B | l μl@10 uM | 25 μl |
| Tube 2-B | 1 μl@10 uM | 25 μl |

10.) Mix diluted mRNA and MessengerMax Transfection reagent as follows: Table 13.

TABLE 13

| Mix | Content |
|---|---|
| Tube 1-A with Tube-1AM | 2^{nd} dose sgRNA |
| Tube 1-B with Tube-1BM | 2^{nd} dose ssODN |
| Tube 2-B with Tube-2BM | 2^{nd} dose ssODN |

Incubate complexes for 5 min.

11.) Remove media, and wash cells with 1 mL of phosphate-buffered saline (PBS) without Ca²⁺ and Mg²⁺ and aspirate.

12.) Add the transfection complexes to each well. Plate Setup below in Table 14.

TABLE 14

| | |
|---|---|
| Well 1 | 2^{nd} dose sgRNA + ssODN |
| Well 2 (negative control) | 2^{nd} dose ssODN |

13.) Add Y27632 supplemented TeSR™-E8™ to each well so final volume is 600 μl. Place plate into low oxygen incubator.

14.) After 4-6 hours, aspirate transfection media, and replace with 2 ml Y27632 supplemented TeSR™-E8™. Allow cells to incubate overnight.

15.) After 2 days, CRISPR treated cells are ready to be:
i. Passaged/Split again.
ii. Analyzed via RT-PCR screening to assess HDR efficiency.
iii. Passaged into single cell for clone screening.

VI. Exemplary method embodiments for Cell Lysis and genomic DNA amplification

Materials
D-PBS
PRG-1 EDTA
TrypLE 1×
Allele Mouse Tail Lysis buffer (150 mM NaCl, 80 mM Tris-HCl pH 8.5, 5 mM EDTA, 2.5 mM MgCl₂, 1% NP40, 1% Triton X100, and 4% Tween 20)
Herculase II Fusion DNA Polymerase Kit
Costar™ Sterile Disposable Reagent Reservoirs
Non-skirted 96-well PCR plate
AlumaSeal CS Sealing Films
Skirted 96-well PCR plate
Surface Bind PCR plate purification kit
NucleoSpin® Gel and PCR Clean-up

a. Lysing bulk cell population (6-well plate)
1.) Wash cells with 1 mL of phosphate-buffered saline (PBS) without Ca²⁺ and Mg²⁺ and aspirate. Note: There is no need to remove regions of differentiated cells.
2.) Add 0.3 mL of PRG-1, then aspirate most of the PRG-1 within 15 s leaving ˜80 μL in the well (so that colonies are exposed to a thin film of liquid).
3.) Incubate at 37° C. for 3-5 minutes.
4.) Tap plate to aid detachment. Add 3 ml of phosphate-buffered saline (PBS) and gently pipette cells from plate bottom and transfer to a 15 ml conical tube. *Optionally: passage a portion (>50,000 Cells) of detached cells onto a new Matrigel coated plate according to protocol V.b. This optional step is performed after a CRISPR transfection and allows for a portion of the population to be grown while the remaining cells are lysed and analyzed. When bulk analysis shows HDR efficiency is suitable, the remaining cells upon reaching confluence can be cloned to single cell by limited dilution (see V.c).
5.) Centrifuge at 300×g for 3 minutes at room temperature to pellet cells. Aspirate PBS.
6.) Resuspend the cell pellet in 150 μl Lysis buffer. Transfer into a PCR tube and run the following program in a thermocycler: 65° C. for 15 min, 68° C. for 15 min, and 95° C. for 15 min.:
7.) After completion of the thermocycler program, the lysate is ready for use as template in PCR reactions.

b.) Lysing clonal populations (96-well plate)
1.) Remove media and wash wells with 100 μl of phosphate-buffered saline (PBS) without Ca²⁺ and Mg²⁺ each and aspirate.
2.) Using a multichannel pipette, add 50 μl of Lysis Buffer directly to the wells. Pipette up and down 4-5 times.
3.) Transfer Lysis buffer from cell culture plate onto a non-skirted PCR plate. Seal the top of the plate with an AlumaSeal. Using a thermocycler, run the following program on the plate −65° C. for 15 min, 68° C. for 15 min, and 95° C.:
4.) After completion of the thermocycler program, lysate is ready to be used in PCR reactions.

c.) Amplifying genomic DNA template from lysate
1.) In PCR tubes on ice, assemble PCR reaction for 6-well plate lysate as follows in Table 15:

TABLE 15

| Component | Volume (μl) |
|---|---|
| Lysate | 1 |
| FWD primer (10 uM) | 2 |
| REV primer (10 uM) | 2 |
| Herculase II 5X Buffer | 8 |
| 10 mM dNTPs | 0.8 |
| Herculase II polymerase | 0.4 |
| H2O | 25.8 |

2.) In a non-skirted PCR plate, assemble PCR reaction for 96-well lysate as follows Table 16.

TABLE 16

| Component | Volume (μl) |
|---|---|
| Lysate | 5 |
| FWD primer (10 uM) | 2 |
| REV primer (10 uM) | 2 |
| Herculase II 5X Buffer | 8 |
| 10 mM dNTPs | 0.8 |
| Herculase II polymerase | 0.4 |
| H₂O | 21.8 |

3.) Run the following PCR program Table 17:

TABLE 17

| 95° C. | 3 | min |
|---|---|---|
| 95° C. | 30 | s |
| Use optimized | 30 | s |
| annealing | | |
| temperature. | | |
| 72° C. | 30 | s |
| 26 cycles | | |
| 72° C. | 5 | min |

| | |
|---|---|
| 10° C. | Hold |

4.) After program has been completed, run 2 μl of PCR product on a 1% agarose gel to confirm amplification and to assess amplification efficiency. Optimization may be needed (annealing temperature, and primer design) when PCR bands display weak intensity. A robust amplification at <26 cycles is needed before proceeding to screening.
5.) Purify PCR product from the bulk lysate template using NucleoSpin kit according to the manufacturer's protocol. For purification of PCR product from 96 well plate lysates, use the SurfaceBind plate purification kit according to the manufacturer's protocol.
6.) Purified amplicon libraries are now suitable for qPCR based screening.

VII. Exemplary method embodiments for in vitro Cas9-sgRNA cleavage assay

Materials:
Recombinant Cas9Wt protein (from III)
In Vitro transcribed sgRNA (from IV)
Cleavage template (Amplicon generated from lysate with sgRNA site)
10×Cas9 Nuclease Reaction Buffer (20 mM HEPES, 100 mM NaCl, 5 mM MgCl₂, 0.1 mM EDTA)

1.) Assemble the reaction at room temperature in the following order as shown in Table 18:

TABLE 18

| Component | 30 μl reaction | |
|---|---|---|
| Nuclease-free water | 22.5 | μl |
| 10X Cas9 Nuclease Reaction Buffer | 3 | μl |
| sgRNA (100 ng/μl) | 0.5 μl (50 ng) | |
| Recombinant Cas9Wt protein (100 μg/ml) | 1 μl (0.1 μg) final) | |
| Reaction volume | 27 | μl |

Pre-incubate for 10 minutes at 25° C.

| 100 nM (33 ng/μl) Substrate DNA | 3 μl (100 ng) | |
|---|---|---|
| Total reaction volume | 30 | μl |

2.) Mix thoroughly and pulse-spin in a microfuge. Then Incubate at 37° C. for 45 minutes

3.) Proceed with fragment analysis by running the sample on a 0.5% to 1% agarose gel.

VIII. Exemplary method embodiments for qPCR based screening

Materials
LightCycler® 480 SYBR Green I Master Mix
MicroAmp® Fast Optical 96-Well Reaction Plate, 0.1 mL
Gibson Assembly Master Mix
DH5α competent cells
Herculase II Fusion DNA Polymerase Kit
QuikChange Site-Directed Mutagenesis Kit
NucleoSpin® Gel and PCR Clean-up
Excel Scientific ThermalSeal® RT™ Films for Real-Time PCR

a.) Construction of plasmids for mutant amplicon copy number standards
1.) Targeted loci are PCR amplified according to protocol outlined in VI.c. Template should be from lysate of non-transfected cells. In this case, forward and reverse primers should also have overlap regions with the pIVT vector for Gibson Assembly.

Example primers (n=loci specific):

Fwd:
(SEQ ID NO: 5)
5′-GAGTAAGAAGAAATATAAGAGCCAC
Cnnnnnnnnnnnnnnnnnn-3′
Rev:
(SEQ ID NO: 6)
5′-AGGCAAGCCCCGCAGAAGGCAGC
nnnnnnnnnnnnnnnnnn-3′

pIVT vector must also be linearized via PCR by using pIVT-F and R

pIVT-F:
(SEQ ID NO: 7)
GCTGCCTTCTGCGGGGCTTGCCT
pIVT-R:
(SEQ ID NO: 8)
GGTGGCTCTTATATTTCTTCTTACTC

2.) Combine insert (genomic loci amplicon) and vector (pIVT backbone) with Gibson assembly mix. Suggested Gibson Assembly setup Table 19.

TABLE 19

| | | |
|---|---|---|
| Gibson MasterMix (in house or from NEB) | 15 | μl |
| pIVT Backbone | 200 | ng |
| CRISPR Target region amplicon | 200 | ng |

| | |
|---|---|
| H₂O | Fill to 20 μl total |

Gibson Assembly reaction is incubated for 1 hr at 50° C., then transformed into DH5α chemical competent cells. The resulting vector will be designated as the wild-type vector.

3.) Using the newly assembled wild type vector as a template, create the intended mutation you wish to screen for in the region of interest with the QuikChange Site-Directed Mutagenesis Kit. Perform site directed mutagenesis according to manufacturer's (Agilent) protocol. The resulting construct will be designated as the mutant vector.

4.) With the completed mutant and wild type pIVT constructs, proceed to making the amplicon standards. Assemble separate PCR reactions using wild type and mutant pIVT constructs as template Table 20.

TABLE 20

| Component | Volume (μl) |
|---|---|
| Mutant or Wild Type Vector | 10 ng |
| M13-F primer (10 μM) | 2 |
| M13-R primer (10 μM) | 2 |
| Phusion GC 5X Buffer | 8 |
| 10 mM dNTPs | 0.8 |
| Herculase II Fusion DNA Polymerase | 0.4 |
| H₂O | 21.8 |

Run the following PCR program as shown in Table 21:

TABLE 21

| 95° C. | 3 | min |
|---|---|---|
| 95° C. | 30 | s |
| 60° C. | 30 | s |
| 72° C. | 30 | s |
| 30 cycles | | |
| 72° C. | 5 | min |

| | |
|---|---|
| 10° C. | Hold |

5.) Run 1 μl of PCR product on a 1% agarose gel to confirm suitable yield and correct size. Then proceed to a NucleoSpin PCR clean-up procedure according to the manufacturers protocol.

6.) Quantify the resulting amplicons. Make dilutions of the amplicon standards so they are both standardized to 60 fg/μl which would correspond to ˜60,000 copies of the amplicon per μl.

7.) With the mutant and wild type standards diluted to a working concentration, make the following wild type:mutant ratios (for use in qPCR assay development) Table 22.

TABLE 22

| Ratio | Mixture components (at 60 fg/μl) |
|---|---|
| 100% wild type (0% mutant) | 100 μl of wild type amplicon |
| 99% wild type (1% mutant) | 99 μl wild type amplicon, |
| | 1 μl mutant amplicon |
| 90% wild type (10% mutant) | 90 μl wild type amplicon, |
| | 10 μl mutant amplicon |
| 50% wild type (50% mutant) | 50 μl wild type amplicon, |
| | 50 μl mutant amplicon |

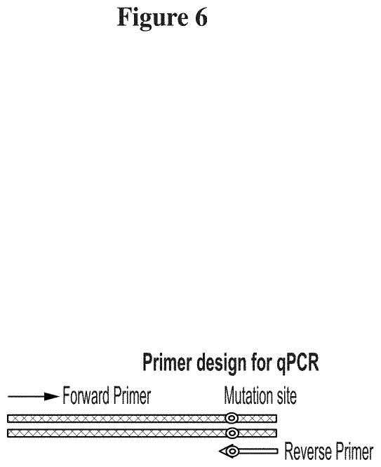

b.) qPCR assay development with mutant amplicon copy number standards
1.) Design a forward and reverse primer pair that is best suited for screening amplicons. Criteria for the design:
≤300 bp product.
Forward primer should be around 200-300 bp upstream of intended mutation.
Reverse primer should have the intended mutation base(s) at the leading 5′ end. (see FIG. 6)

On ice, prepare the qPCR reaction in a Fast Optical 96-Well Reaction Plate as shown in Table 23:

TABLE 23

| | |
|---|---|
| SYBR Green I Master Mix | 7.5 μl |
| Forward Primer | 1.0 μl |
| Reverse Primer | 1.0 μl |
| H2O | 0.5 μl |
| Amplicon Standard Template | 5.0 μl (300 fg) |

Note:
In certain embodiments, because multiple reactions are assembled, it is best to prepare a master mix with SYBR Green, primer pair, and H₂O, then add amplicon standard template at the last step.

Assign the amplicon standard template in the following orientation as shown in Table 24.

TABLE 24

| | 1 | | 2 | |
|---|---|---|---|---|
| A | 0% mutant | (100% wild type) | 10% mutant | (90% wild type) |
| B | 0% mutant | (100% wild type) | 50% mutant | (50% wild type ) |
| C | 0% mutant | (100% wild type) | 50% mutant | (50% wild type ) |
| D | 1% mutant | (99% wild type) | 50% mutant | (50% wild type ) |
| E | 1% mutant | (99% wild type) | 100% mutant | (0% wild type) |
| F | 1% mutant | (99% wild type) | 100% mutant | (0% wild type) |
| G | 10% mutant | (90% wild type) | 100% mutant | (0% wild type) |

| | | |
|---|---|---|
| H | 10% mutant | (90% wild type) |

3.) Once plate is prepared, seal with transparent ThermalSeal and perform a quick spin (˜3 g for 10 s). Place back on ice while setting up the software program.
4.) Running the software:
   i. Open StepOne Plus software, and log in as “GUEST”.
   ii. Open Template file by clicking on “Template” Button on the bottom left, and select the “Crispr-Standards” template file. (D:\Applied Biosystems\StepOne Software v2.3\config\templates)
   iii. Go to the “Experiment Properties” page and fill in the “Experiment Name” with an appropriate title. (E.g. Crispr_standards_test12-25-18)
   iv. Next go to the “Run Method” page, and change annealing temperature to a temperature that is optimal for the experiment.
   v. Place the prepared plate into the StepOnePlus machine and shut the drawer/cover. Click the green “Start Run” button to initiate the run.

c.) Analysis of amplification curves.
1.) To confirm that the primer design is effectively discriminating against amplification of wild type, compare Ct values of the different ratios.
2.) Dose response should be observed: Ct values should become left shifted (smaller) with increasing ratios of mutant population.
3.) Some suggested ΔCt values for each standard point shown in Table 25:

TABLE 25

| | |
|---|---|
| Ratio | ΔCt |

| | | |
|---|---|---|
| 0% mutant | (100% wild type) | 0 |
| 1% mutant | (99% wild type) | ≥3 |
| 10% mutant | (90% wild type) | ≥6 |
| 50% mutant | (50% wild type ) | ≥8 |

Example Amplification Ct curves (see FIG. 7)
4.) When Amplicon standards produce suitable results (as exemplified above), proceed to screening of CRISPR edited cells.

d.) Screening of bulk population
1.) With the amplicons produced in step VI.c, standardize the concentration down to 60 fg/ul.
2.) On ice, prepare the qPCR reaction as follows in a Fast Optical 96-Well Reaction Plate as shown in Table 26:

TABLE 26

| | |
|---|---|
| SYBR Green I Master Mix | 7.5 μl |
| Forward Primer | 1.0 μl |
| Reverse Primer | 1.0 μl |
| H₂O | 0.5 μl |
| Amplicons from experiment or | 5.0 μl (300 fg) |
| Amplicon standards | |

Note:
because multiple reactions are assembled, it is best to prepare a master mix with SYBR Green, primer pair and H₂O, then add amplicon template at the last step.

3.) Assign amplicon standards in the orientation outlined in step VIII.b.2. A. In triplicate, assign the standardized amplicon libraries from bulk population into the plate. Make sure to include a negative control (ssODN only transfected cells).
4.) Once plate is prepared, seal with transparent ThermalSeal and perform a quick spin (˜3 g for 10 s). Place back on ice while setting up the software program.
5.) Run software as outlined in step VIII.b.4. Before starting, make sure to assign CRISPR amplicon libraries and negative control from the experiment according to how they were allocated in the reaction plate in the “Plate Setup” window. Run program when everything is setup properly.

e.) Analysis of Bulk Screen
1.) With the qPCR results from VIII.d, amplicon libraries can be compared with each other (CRISPR treated cells and non transfected cells) and with the standards. ΔCt for CRISPR treated cells should be calculated by taking the negative control Ct value and subtracting the CRISPR treated cells Ct value. CRISPR treated cells should show a ΔCt comparable to the 1% standard. (Note: it has been observed that the ΔCt increases after one passage).
2.) When the ΔCt for bulk population is ˜1%, proceed to single cell cloning procedure as outlined in step V

f.) qPCR screening of clones from 96-well plate
1.) The SurfaceBind purified clonal amplicon library plate (as described in step VI.c.5) will be used as the template for qPCR screening.
2.) Perform a 1:1000 dilution of the clonal amplicon libraries in a 96-well 2 ml collection plate. Use AlumaSeal to seal the plate and vortex to mix.
3.) On ice, prepare the qPCR reaction as follows in a Fast Optical 96-Well Reaction Plate as shown in Table 27:

TABLE 27

| | |
|---|---|
| SYBR Green I Master Mix | 7.5 μl |
| Forward Primer | 1.0 μl |
| Reverse Primer | 1.0 μl |
| H₂O | 0.5 μl |
| 1:1000 diluted amplicon library | 5.0 μl |

Note:
because multiple reactions are carried out, prepare a master mix containing SYBR Green, primer pair and H₂O, then add the diluted amplicon library last. Be sure to preserve the plate orientation.

4.) Once the plate is prepared, seal it with transparent ThermalSeal and perform a quick spin (˜3 g for 10 s). Place back on ice while setting up the software program.
5.) Run software as outlined in step VIII.b.4 with the “96_well_screen” template file (D:\Applied Biosystems\StepOne Software v2.3\config\templates). (Make sure the “Run Setup” window and it's parameters are identical with those in the bulk qPCR screen assay).

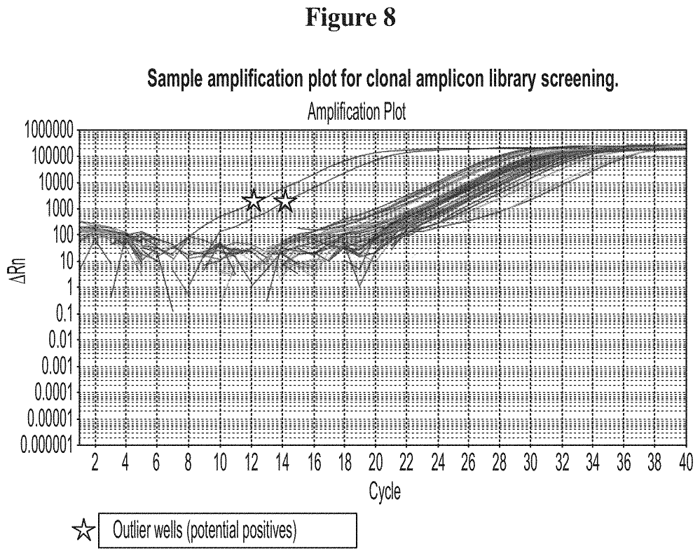

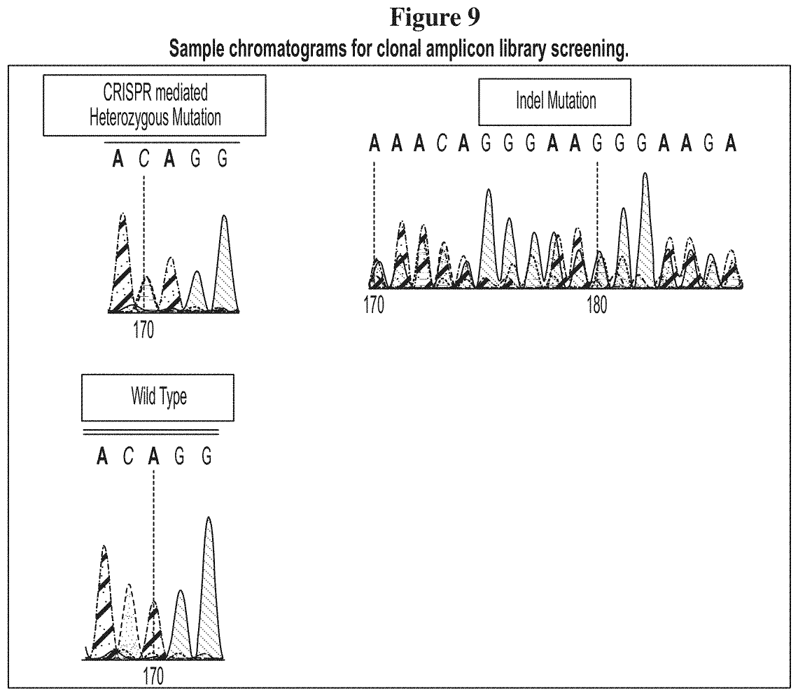

g.) Analysis of clonal qPCR screening.
1.) qPCR screening of clonal amplicon libraries commonly result in high variation, however given a bulk population that has an HDR efficiency of ˜1%, there will be 1-3 low Ct outlier wells. See below for sample data: (see FIG. 8)
2.) Once left shifted Ct outliers are identified, expand the corresponding wells in the duplicate plate according to the protocol outlined in V.e.
3.) Once selected wells are expanded and confluent, lyse a portion of the cells and prepare amplicon libraries. Send these to be sequenced via Sanger Sequencing. Analyze the intended mutation site in the chromatogram results. Heterozygous mutations will show dual peaks at intended site, while homozygous will have only the mutant base pair peak. Mixed population of edited and unedited cells may also show up as dual peaks. Furthermore, CRISPR-mediated insertions and deletions (indels) will produce additional peaks throughout the region proximal to the PAM site. A detailed analysis of the chromatogram is necessary for understanding the genetics of the cell population. See FIG. 9

SEQUENCE LISTING
SEQ ID NO: 1:
cas9_wt
atggccccaaagaaaaagcggaaggtcggtatcca
cggagtcccagcagccgacaagaaatacagcatcg
gcctggacatcggcaccaactctgtgggctgggcc
gtgatcaccgacgagtacaaggtgcccagcaagaa
attcaaggtgctgggcaacaccgacagacacagca
tcaagaagaacctgatcggagccctgctgacgaca
gcggcgaaacagccgaggccacccggctgaagaga
accgccagacggagatacaccagacggaagaaccg
gatctgctatctgcaagagatatcagcaacgagat
ggccaaggtggacgacagcttatccacagactgga
agagtccacctggtggaagaggataagaagcacga
gcggcaccccatcacggcaacatcgtggacgaggt
ggcctaccacgagaagtaccccaccatctaccacc
tgagaaagaaactggtggacagcaccgacaaggcc
gacctgcggctgatctatctggccctggcccacat
gatcaagttccggggccacttcctgatcgagggcg
acctgaaccccgacaacagcgacgtggacaagctg
ttcatccagctggtgcagacctacaaccagctgac
gaggaaaaccccatcaacgccagcggcgtggacgc
caaggccatcctgtctgccagactgagcaaaagca
gacggctggaaaatctgatcgcccagctgcccggc
gagaagaagaatggcctgttcggcaacctgattgc
cctgagcctgggcctgacccccaacttcaagagca
acttcgacctggccgaggatgccaaactgcagctg
agcaaggacacctacgacgacgacctggacaacct
gctggcccagatcggcgaccagtacgccgacctga
tctggccgccaagaacctgtccgacgccatcctgc
tgagcgacatcctgagagtgaacaccgagatcacc
aaagccccactgagcgcctctatgatcaagagata
cgacgagcaccaccaggacctgaccctgctgaaag
ctctcgtgcggcagcagctgcctgagaagtacaaa
gagattacttcgaccagagcaagaacggctacgcc
ggctacattgacggcggagccagccaggaagagac
tacaagttcatcaagcccatcctggaaaagatgga
cggcaccgaggaactgctcgtgaagctgaacagag
aggacctgctgcggaagcagcggaccttcgacaac
ggcagcatcccccaccagatccacctgggagagct
gcacgccattctgcggcggcaggaagattataccc
attcctgaaggacaaccgggaaaagatcgagaaga
tcctgaccaccgcatcccctactacgtgggccctc
tggccaggggaaacagcagattcgcctggatgacc
agaaagagcgaggaaaccatcaccccctggaactt
cgaggaagtggtggacaagggcgcaccgcccagag
cttcatcgagagaatgaccaacttcgataagaacc
tgcccaacgagaaggtgctgcccaagcacagcctg
ctgtacgagtacttcaccgtgtataacgagctgac
caaagtgaaatacgtgaccgagggaatgagaaagc
ccgccacctgagcggcgagcagaaaaaggccatcg
tggacctgctgttcaagaccaacaggaaagtgacc
gtgaagcagctgaaagaggactacttcaagaaaat
cgagtgcttcgactccgtggaaatctccggcgtgg
aagatcggttcaacgcctccctgggcacataccac
gatctgctgaaaattatcaaggacaaggacttcct
ggacaatgaggaaaacgaggacattctggaagata
tcgtgctgaccctgacactgatgaggacagagaga
tgatcgaggaacggctgaaaacctatgcccacctg
acgacgacaaagtgatgaagcagctgaagcggcgg
agatacaccggctggggcaggctgagccggaagct
gatcaacggcatccgggacaagcagtccggcaaga
caatcctggatttcctgaagtccgacggcttcgcc
aacagaaacttcatgcagctgatccacgacgacag
cctgaccataaagaggacatccagaaagcccaggt
gtccggccagggcgatagcctgcacgagcacattg
ccaatctggccggcagccccgccattaagaagggc
atcctgcagacagtgaaggtggtggacgagctcgt
gaaagtgatgggccggcacaagcccgagaacatcg
tgatcgaaatggccagagagaaccagaccacccag
aagggacagaagaacagccgcgagagaatgaagcg
gatcgaagagggcatcaaagagctgggcagccaga
tcctgaaagaacaccccgtggaaaacacccagctg
cagaacgagaagctgtacctgtactacctgcagaa
tgggcgggatatgtacgtggaccaggaactggaca
tcaaccggctgtccgactacgatgtggaccacatc
gtgcctcagagctttctgaaggacgactccatcga
caacaaggtgctgaccagaagcgacaagaaccggg
gcaagagcgacaacgtgccctccgaagaggtcgtg
aagaagatgaagaactactggcggcagctgctgaa
cgccaaactgattacccagagaaagacgacaatct
gaccaaggccgagagaggcggcctgagcgaactgg
ataaggccggcttcatcaagagacagctggtggaa
acccggcagatcacaaagcacgtggcacagatcct
ggactcccggatgaacactaagtacgacgagaatg
acaagctgatccgggaagtgaaagtgatcaccctg
aagtccaagctggtgtccgataccggaaggatacc
agattacaaagtgcgcgagatcaacaactaccatc
acgcccatgacgcctacctgaacgccgtcgtggga
accgccctgatcaaaaagtaccctaagctggaaag
cgagttcgtgtacggcgactacaaggtgtacgacg
tgcggaagatgatcgccaagagcgagcaggaaatc
ggcaaggctaccgccaagtacttcactacagcaac
atcatgaactttttcaagaccgagattaccctggc
caacggcgagatccggaagcggcctctgatcgaga
caaacggcgaaaccggggagatcgtgtgggataag
ggccgggattagccaccgtgcggaaagtgctgagc
atgccccaagtgaatatcgtgaaaaagaccgaggt
gcagacaggcggcttcagcaaagagtctatcctgc
ccaagaggaacagcgataagctgatcgccagaaag
aaggactgggaccctaagaagtacggcggcttcga
cagccccaccgtggcctattctgtgctggtggtgg
ccaaagtggaaaagggcaagtccaagaaactgaag
agtgtgaaagagctgctggggatcaccatcatgga
aagaagcagcttcgagaagaatcccatcgactact
ggaagccaagggctacaaagaagtgaaaaaggacc
tgatcatcaagctgcctaagtactccctgttcgag
ctggaaaacggccggaagagaatgctggcctctgc
cggcgaactgcagaagggaaacgaactggccctgc
cctccaaatatgtgaacttcctgtacctggccagc
cactatgagaagctgaagggctcccccgaggataa
tgagcagaaacagctgatgtggaacagcataagca
ctacctggacgagatcatcgagcagatcagcgagt
tctccaagagagtgatcctggccgacgctaatctg
gacaaagtgctgtccgcctacaacaagcatcggga
taagcccatcagagagcaggccgagaatatcatcc
acctgataccctgaccaatctgggagcccctgccg
catcaagtactagacaccaccatcgaccggaagag
gtacaccagcaccaaagaggtgctggacgccaccc
tgatccaccagagcatcaccggcctgtacgagaca
cggatcgacctgtctcagctgggaggtgacaagcg
tcctgctgctactaagaaagctggtcaagctaaga
aaaagaaatga
SEQ ID NO: 2
cas9_D 10A
atggccccaaagaaaaagcggaaggtcggtatcca
cggagtcccagcagccgacaagaaatacagcatcg
gcctggCcatcggcaccaactctgtgggctgggcc
gtgatcaccgacgagtacaaggtgcccagcaagaa
attcaaggtgctgggcaacaccgacagacacagca
tcaagaagaacctgatcggagccctgctgacgaca
gcggcgaaacagccgaggccacccggctgaagaga
accgccagacggagatacaccagacggaagaaccg
gatctgctatctgcaagagatatcagcaacgagat
ggccaaggtggacgacagcttatccacagactgga
agagtccacctggtggaagaggataagaagcacga
gcggcaccccatcacggcaacatcgtggacgaggt
ggcctaccacgagaagtaccccaccatctaccacc
tgagaaagaaactggtggacagcaccgacaaggcc
gacctgcggctgatctatctggccctggcccacat
gatcaagttccggggccacttcctgatcgagggcg
acctgaaccccgacaacagcgacgtggacaagctg
ttcatccagctggtgcagacctacaaccagctgac
gaggaaaaccccatcaacgccagcggcgtggacgc
caaggccatcctgtctgccagactgagcaaaagca
gacggctggaaaatctgatcgcccagctgcccggc
gagaagaagaatggcctgttcggcaacctgattgc
cctgagcctgggcctgacccccaacttcaagagca
acttcgacctggccgaggatgccaaactgcagctg
agcaaggacacctacgacgacgacctggacaacct
gctggcccagatcggcgaccagtacgccgacctga
tctggccgccaagaacctgtccgacgccatcctgc
tgagcgacatcctgagagtgaacaccgagatcacc
aaagccccactgagcgcctctatgatcaagagata
cgacgagcaccaccaggacctgaccctgctgaaag
ctctcgtgcggcagcagctgcctgagaagtacaaa
gagattacttcgaccagagcaagaacggctacgcc
ggctacattgacggcggagccagccaggaagagac
tacaagttcatcaagcccatcctggaaaagatgga
cggcaccgaggaactgctcgtgaagctgaacagag
aggacctgctgcggaagcagcggaccttcgacaac
ggcagcatcccccaccagatccacctgggagagct
gcacgccattctgcggcggcaggaagattataccc
attcctgaaggacaaccgggaaaagatcgagaaga
tcctgaccaccgcatcccctactacgtgggccctc
tggccaggggaaacagcagattcgcctggatgacc
agaaagagcgaggaaaccatcaccccctggaactt
cgaggaagtggtggacaagggcgcaccgcccagag
cttcatcgagagaatgaccaacttcgataagaacc
tgcccaacgagaaggtgctgcccaagcacagcctg
ctgtacgagtacttcaccgtgtataacgagctgac
caaagtgaaatacgtgaccgagggaatgagaaagc
ccgccacctgagcggcgagcagaaaaaggccatcg
tggacctgctgttcaagaccaacaggaaagtgacc
gtgaagcagctgaaagaggactacttcaagaaaat
cgagtgcttcgactccgtggaaatctccggcgtgg
aagatcggttcaacgcctccctgggcacataccac
gatctgctgaaaattatcaaggacaaggacttcct
ggacaatgaggaaaacgaggacattctggaagata
tcgtgctgaccctgacactgtttgaggacagagag
atgatcgaggaacggctgaaaacctatgcccacct
gttcgacgacaaagtgatgaagcagctgaagcggc
ggagatacaccggctggggcaggctgagccggaag
ctgatcaacggcatccgggacaagcagtccggcaa
gacaatcctggatttcctgaagtccgacggcttcg
ccaacagaaacttcatgcagctgatccacgacgac
agcctgaccataaagaggacatccagaaagcccag
gtgtccggccagggcgatagcctgcacgagcacat
tgccaatctggccggcagccccgccattaagaagg
gcatcctgcagacagtgaaggtggtggacgagctc
gtgaaagtgatgggccggcacaagcccgagaacat
cgtgatcgaaatggccagagagaaccagaccaccc
agaagggacagaagaacagccgcgagagaatgaag
cggatcgaagagggcatcaaagagctgggcagcca
gatcctgaaagaacaccccgtggaaaacacccagc
tgcagaacgagaagctgtacctgtactacctgcag
aatgggcgggatatgtacgtggaccaggaactgga
catcaaccggctgtccgactacgatgtggaccaca
tcgtgcctcagagctttctgaaggacgactccatc
gacaacaaggtgctgaccagaagcgacaagaaccg
gggcaagagcgacaacgtgccctccgaagaggtcg
tgaagaagatgaagaactactggcggcagctgctg
aacgccaaactgattacccagagaaagacgacaat
ctgaccaaggccgagagaggcggcctgagcgaact
ggataaggccggcttcatcaagagacagctggtgg
aaacccggcagatcacaaagcacgtggcacagatc
ctggactcccggatgaacactaagtacgacgagaa
tgacaagctgatccgggaagtgaaagtgatcaccc
tgaagtccaagctggtgtccgataccggaaggata
ccagattacaaagtgcgcgagatcaacaactacca
tcacgcccatgacgcctacctgaacgccgtcgtgg
gaaccgccctgatcaaaaagtaccctaagctggaa
agcgagttcgtgtacggcgactacaaggtgtacga
cgtgcggaagatgatcgccaagagcgagcaggaaa
tcggcaaggctaccgccaagtacttcactacagca
acatcatgaactttttcaagaccgagattaccctg
gccaacggcgagatccggaagcggcctctgatcga
gacaaacggcgaaaccggggagatcgtgtgggata
agggccgggattagccaccgtgcggaaagtgctga
gcatgccccaagtgaatatcgtgaaaaagaccgag
gtgcagacaggcggcttcagcaaagagtctatcct
gcccaagaggaacagcgataagctgatcgccagaa
agaaggactgggaccctaagaagtacggcggcttc
gacagccccaccgtggcctattctgtgctggtggt
ggccaaagtggaaaagggcaagtccaagaaactga
agagtgtgaaagagctgctggggatcaccatcatg
gaaagaagcagcttcgagaagaatcccatcgacta
ctggaagccaagggctacaaagaagtgaaaaagga
cctgatcatcaagctgcctaagtactccctgttcg
agctggaaaacggccggaagagaatgctggcctct
gccggcgaactgcagaagggaaacgaactggccct
gccctccaaatatgtgaacttcctgtacctggcca
gccactatgagaagctgaagggctcccccgaggat
aatgagcagaaacagctgatgtggaacagcataag
cactacctggacgagatcatcgagcagatcagcga
gttctccaagagagtgatcctggccgacgctaatc
tggacaaagtgctgtccgcctacaacaagcatcgg
gataagcccatcagagagcaggccgagaatatcat
ccacctgataccctgaccaatctgggagcccctgc
cgcatcaagtactagacaccaccatcgaccggaag
aggtacaccagcaccaaagaggtgctggacgccac
cctgatccaccagagcatcaccggcctgtacgaga
cacggatcgacctgtctcagctgggaggtgacaag
cgtcctgctgctactaagaaagctggtcaagctaa
gaaaaagaaatga
SEQ ID NO: 3
cas9_H840A
atggccccaaagaaaaagcggaaggtcggtatcca
cggagtcccagcagccgacaagaaatacagcatcg
gcctggacatcggcaccaactctgtgggctgggcc
gtgatcaccgacgagtacaaggtgcccagcaagaa
attcaaggtgctgggcaacaccgacagacacagca
tcaagaagaacctgatcggagccctgctgacgaca
gcggcgaaacagccgaggccacccggctgaagaga
accgccagacggagatacaccagacggaagaaccg
gatctgctatctgcaagagatatcagcaacgagat
ggccaaggtggacgacagcttatccacagactgga
agagtccacctggtggaagaggataagaagcacga
gcggcaccccatcacggcaacatcgtggacgaggt
ggcctaccacgagaagtaccccaccatctaccacc
tgagaaagaaactggtggacagcaccgacaaggcc
gacctgcggctgatctatctggccctggcccacat
gatcaagttccggggccacttcctgatcgagggcg
acctgaaccccgacaacagcgacgtggacaagctg
ttcatccagctggtgcagacctacaaccagctgac
gaggaaaaccccatcaacgccagcggcgtggacgc
caaggccatcctgtctgccagactgagcaaaagca
gacggctggaaaatctgatcgcccagctgcccggc
gagaagaagaatggcctgttcggcaacctgattgc
cctgagcctgggcctgacccccaacttcaagagca
acttcgacctggccgaggatgccaaactgcagctg
agcaaggacacctacgacgacgacctggacaacct
gctggcccagatcggcgaccagtacgccgacctga
tctggccgccaagaacctgtccgacgccatcctgc
tgagcgacatcctgagagtgaacaccgagatcacc
aaagccccactgagcgcctctatgatcaagagata
cgacgagcaccaccaggacctgaccctgctgaaag
ctctcgtgcggcagcagctgcctgagaagtacaaa
gagattacttcgaccagagcaagaacggctacgcc
ggctacattgacggcggagccagccaggaagagac
tacaagttcatcaagcccatcctggaaaagatgga
cggcaccgaggaactgctcgtgaagctgaacagag
aggacctgctgcggaagcagcggaccttcgacaac
ggcagcatcccccaccagatccacctgggagagct
gcacgccattctgcggcggcaggaagattataccc
attcctgaaggacaaccgggaaaagatcgagaaga
tcctgaccaccgcatcccctactacgtgggccctc
tggccaggggaaacagcagattcgcctggatgacc
agaaagagcgaggaaaccatcaccccctggaactt
cgaggaagtggtggacaagggcgcaccgcccagag
cttcatcgagagaatgaccaacttcgataagaacc
tgcccaacgagaaggtgctgcccaagcacagcctg
ctgtacgagtacttcaccgtgtataacgagctgac
caaagtgaaatacgtgaccgagggaatgagaaagc
ccgccacctgagcggcgagcagaaaaaggccatcg
tggacctgctgttcaagaccaacaggaaagtgacc
gtgaagcagctgaaagaggactacttcaagaaaat
cgagtgcttcgactccgtggaaatctccggcgtgg
aagatcggttcaacgcctccctgggcacataccac
gatctgctgaaaattatcaaggacaaggacttcct
ggacaatgaggaaaacgaggacattctggaagata
tcgtgctgaccctgacactgatgaggacagagaga
tgatcgaggaacggctgaaaacctatgcccacctg
acgacgacaaagtgatgaagcagctgaagcggcgg
agatacaccggctggggcaggctgagccggaagct
gatcaacggcatccgggacaagcagtccggcaaga
caatcctggatttcctgaagtccgacggcttcgcc
aacagaaacttcatgcagctgatccacgacgacag
cctgaccataaagaggacatccagaaagcccaggt
gtccggccagggcgatagcctgcacgagcacattg
ccaatctggccggcagccccgccattaagaagggc
atcctgcagacagtgaaggtggtggacgagctcgt
gaaagtgatgggccggcacaagcccgagaacatcg
tgatcgaaatggccagagagaaccagaccacccag
aagggacagaagaacagccgcgagagaatgaagcg
gatcgaagagggcatcaaagagctgggcagccaga
tcctgaaagaacaccccgtggaaaacacccagctg
cagaacgagaagctgtacctgtactacctgcagaa
tgggcgggatatgtacgtggaccaggaactggaca
tcaaccggctgtccgactacgatgtggacGCcatc
gtgcctcagagctttctgaaggacgactccatcga
caacaaggtgctgaccagaagcgacaagaaccggg
gcaagagcgacaacgtgccctccgaagaggtcgtg
aagaagatgaagaactactggcggcagctgctgaa
cgccaaactgattacccagagaaagacgacaatct
gaccaaggccgagagaggcggcctgagcgaactgg
ataaggccggcttcatcaagagacagctggtggaa
acccggcagatcacaaagcacgtggcacagatcct
ggactcccggatgaacactaagtacgacgagaatg
acaagctgatccgggaagtgaaagtgatcaccctg
aagtccaagctggtgtccgataccggaaggatacc
agattacaaagtgcgcgagatcaacaactaccatc
acgcccatgacgcctacctgaacgccgtcgtggga
accgccctgatcaaaaagtaccctaagctggaaag
cgagttcgtgtacggcgactacaaggtgtacgacg
tgcggaagatgatcgccaagagcgagcaggaaatc
ggcaaggctaccgccaagtacttcactacagcaac
atcatgaactttttcaagaccgagattaccctggc
caacggcgagatccggaagcggcctctgatcgaga
caaacggcgaaaccggggagatcgtgtgggataag
ggccgggattagccaccgtgcggaaagtgctgagc
atgccccaagtgaatatcgtgaaaaagaccgaggt
gcagacaggcggcttcagcaaagagtctatcctgc
ccaagaggaacagcgataagctgatcgccagaaag
aaggactgggaccctaagaagtacggcggcttcga
cagccccaccgtggcctattctgtgctggtggtgg
ccaaagtggaaaagggcaagtccaagaaactgaag
agtgtgaaagagctgctggggatcaccatcatgga
aagaagcagcttcgagaagaatcccatcgactttc
tggaagccaagggctacaaagaagtgaaaaaggac
ctgatcatcaagctgcctaagtactccctgttcga
gctggaaaacggccggaagagaatgctggcctctg
ccggcgaactgcagaagggaaacgaactggccctg
ccctccaaatatgtgaacttcctgtacctggccag
ccactatgagaagctgaagggctcccccgaggata
atgagcagaaacagctgatgtggaacagcataagc
actacctggacgagatcatcgagcagatcagcgag
ttctccaagagagtgatcctggccgacgctaatct
ggacaaagtgctgtccgcctacaacaagcatcggg
ataagcccatcagagagcaggccgagaatatcatc
cacctgataccctgaccaatctgggagcccctgcc
gcatcaagtactagacaccaccatcgaccggaaga
ggtacaccagcaccaaagaggtgctggacgccacc
ctgatccaccagagcatcaccggcctgtacgagac
acggatcgacctgtctcagctgggaggtgacaagc
gtcctgctgctactaagaaagctggtcaagctaag
aaaaagaaatga
cas9_D_10A_H840A
SEQ ID NO: 4
atggccccaaagaaaaagcggaaggtcggtatcca
cggagtcccagcagccgacaagaaatacagcatcg
gcctggCcatcggcaccaactctgtgggctgggcc
gtgatcaccgacgagtacaaggtgcccagcaagaa
attcaaggtgctgggcaacaccgacagacacagca
tcaagaagaacctgatcggagccctgctgacgaca
gcggcgaaacagccgaggccacccggctgaagaga
accgccagacggagatacaccagacggaagaaccg
gatctgctatctgcaagagatatcagcaacgagat
ggccaaggtggacgacagcttatccacagactgga
agagtccacctggtggaagaggataagaagcacga
gcggcaccccatcacggcaacatcgtggacgaggt
ggcctaccacgagaagtaccccaccatctaccacc
tgagaaagaaactggtggacagcaccgacaaggcc
gacctgcggctgatctatctggccctggcccacat
gatcaagttccggggccacttcctgatcgagggcg
acctgaaccccgacaacagcgacgtggacaagctg
ttcatccagctggtgcagacctacaaccagctgac
gaggaaaaccccatcaacgccagcggcgtggacgc
caaggccatcctgtctgccagactgagcaaaagca
gacggctggaaaatctgatcgcccagctgcccggc
gagaagaagaatggcctgttcggcaacctgattgc
cctgagcctgggcctgacccccaacttcaagagca
acttcgacctggccgaggatgccaaactgcagctg
agcaaggacacctacgacgacgacctggacaacct
gctggcccagatcggcgaccagtacgccgacctga
tctggccgccaagaacctgtccgacgccatcctgc
tgagcgacatcctgagagtgaacaccgagatcacc
aaagccccactgagcgcctctatgatcaagagata
cgacgagcaccaccaggacctgaccctgctgaaag
ctctcgtgcggcagcagctgcctgagaagtacaaa
gagattacttcgaccagagcaagaacggctacgcc
ggctacattgacggcggagccagccaggaagagac
tacaagttcatcaagcccatcctggaaaagatgga
cggcaccgaggaactgctcgtgaagctgaacagag
aggacctgctgcggaagcagcggaccttcgacaac
ggcagcatcccccaccagatccacctgggagagct
gcacgccattctgcggcggcaggaagattataccc
attcctgaaggacaaccgggaaaagatcgagaaga
tcctgaccaccgcatcccctactacgtgggccctc
tggccaggggaaacagcagattcgcctggatgacc
agaaagagcgaggaaaccatcaccccctggaactt
cgaggaagtggtggacaagggcgcaccgcccagag
cttcatcgagagaatgaccaacttcgataagaacc
tgcccaacgagaaggtgctgcccaagcacagcctg
ctgtacgagtacttcaccgtgtataacgagctgac
caaagtgaaatacgtgaccgagggaatgagaaagc
ccgccacctgagcggcgagcagaaaaaggccatcg
tggacctgctgttcaagaccaacaggaaagtgacc
gtgaagcagctgaaagaggactacttcaagaaaat
cgagtgcttcgactccgtggaaatctccggcgtgg
aagatcggttcaacgcctccctgggcacataccac
gatctgctgaaaattatcaaggacaaggacttcct
ggacaatgaggaaaacgaggacattctggaagata
tcgtgctgaccctgacactgtttgaggacagagag
atgatcgaggaacggctgaaaacctatgcccacct
gttcgacgacaaagtgatgaagcagctgaagcggc
ggagatacaccggctggggcaggctgagccggaag
ctgatcaacggcatccgggacaagcagtccggcaa
gacaatcctggatttcctgaagtccgacggcttcg
ccaacagaaacttcatgcagctgatccacgacgac
agcctgaccataaagaggacatccagaaagcccag
gtgtccggccagggcgatagcctgcacgagcacat
tgccaatctggccggcagccccgccattaagaagg
gcatcctgcagacagtgaaggtggtggacgagctc
gtgaaagtgatgggccggcacaagcccgagaacat
cgtgatcgaaatggccagagagaaccagaccaccc
agaagggacagaagaacagccgcgagagaatgaag
cggatcgaagagggcatcaaagagctgggcagcca
gatcctgaaagaacaccccgtggaaaacacccagc
tgcagaacgagaagctgtacctgtactacctgcag
aatgggcgggatatgtacgtggaccaggaactgga
catcaaccggctgtccgactacgatgtggacGCca
tcgtgcctcagagctttctgaaggacgactccatc
gacaacaaggtgctgaccagaagcgacaagaaccg
gggcaagagcgacaacgtgccctccgaagaggtcg
tgaagaagatgaagaactactggcggcagctgctg
aacgccaaactgattacccagagaaagacgacaat
ctgaccaaggccgagagaggcggcctgagcgaact
ggataaggccggcttcatcaagagacagctggtgg
aaacccggcagatcacaaagcacgtggcacagatc
ctggactcccggatgaacactaagtacgacgagaa
tgacaagctgatccgggaagtgaaagtgatcaccc
tgaagtccaagctggtgtccgataccggaaggata
ccagattacaaagtgcgcgagatcaacaactacca
tcacgcccatgacgcctacctgaacgccgtcgtgg
gaaccgccctgatcaaaaagtaccctaagctggaa
agcgagttcgtgtacggcgactacaaggtgtacga
cgtgcggaagatgatcgccaagagcgagcaggaaa
tcggcaaggctaccgccaagtacttcactacagca
acatcatgaactttttcaagaccgagattaccctg
gccaacggcgagatccggaagcggcctctgatcga
gacaaacggcgaaaccggggagatcgtgtgggata
agggccgggattagccaccgtgcggaaagtgctga
gcatgccccaagtgaatatcgtgaaaaagaccgag
gtgcagacaggcggcttcagcaaagagtctatcct
gcccaagaggaacagcgataagctgatcgccagaa
agaaggactgggaccctaagaagtacggcggcttc
gacagccccaccgtggcctattctgtgctggtggt
ggccaaagtggaaaagggcaagtccaagaaactga
agagtgtgaaagagctgctggggatcaccatcatg
gaaagaagcagcttcgagaagaatcccatcgacta
ctggaagccaagggctacaaagaagtgaaaaagga
cctgatcatcaagctgcctaagtactccctgttcg
agctggaaaacggccggaagagaatgctggcctct
gccggcgaactgcagaagggaaacgaactggccct
gccctccaaatatgtgaacttcctgtacctggcca
gccactatgagaagctgaagggctcccccgaggat
aatgagcagaaacagctgatgtggaacagcataag
cactacctggacgagatcatcgagcagatcagcga
gttctccaagagagtgatcctggccgacgctaatc
tggacaaagtgctgtccgcctacaacaagcatcgg
gataagcccatcagagagcaggccgagaatatcat
ccacctgataccctgaccaatctgggagcccctgc
cgcatcaagtactagacaccaccatcgaccggaag
aggtacaccagcaccaaagaggtgctggacgccac
cctgatccaccagagcatcaccggcctgtacgaga
cacggatcgacctgtctcagctgggaggtgacaag
cgtcctgctgctactaagaaagctggtcaagctaa
gaaaaagaaatga

### REFERENCES

Cartwright, E. J. (2009). Large-scale mouse mutagenesis. Methods in molecular biology (Clifton, N.J. 561, 275-283.
Cheng, A. W., Wang, H., Yang, H., Shi, L., Katz, Y., Theunissen, T. W., Rangarajan, S., Shivalila, C. S., Dadon, D. B., and Jaenisch, R. (2013). Multiplexed activation of endogenous genes by CRISPR-on, an RNA-guided transcriptional activator system. Cell research 23, 1163-1171.
Cho, S. W., Kim, S., Kim, J. M., and Kim, J. S. (2013). Targeted genome engineering in human cells with the Cas9 RNA-guided endonuclease. Nature biotechnology 31, 230-232.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science (New York, N.Y. 339, 819-823.
DiCarlo, J. E., Norville, J. E., Mali, P., Rios, X., Aach, J., and Church, G. M. (2013). Genome engineering in Saccharomyces cerevisiae using CRISPR-Cas systems. Nucleic acids research 41, 4336-4343.
Fonfara, I., Le Rhun, A., Chylinski, K., Makarova, K. S., Lecrivain, A. L., Bzdrenga, J., Koonin, E. V., and Charpentier, E. (2013). Phylogeny of Cas9 determines functional exchangeability of dual-RNA and Cas9 among orthologous type II CRISPR-Cas systems. Nucleic acids research.
Friedland, A. E., Tzur, Y. B., Esvelt, K. M., Colaiacovo, M. P., Church, G. M., and Calarco, J. A. (2013). Heritable genome editing in C. elegans via a CRISPR-Cas9 system. Nature methods 10, 741-743.
Fu, Y., Foden, J. A., Khayter, C., Maeder, M. L., Reyon, D., Joung, J. K., and Sander, J. D. (2013). High-frequency off-target mutagenesis induced by CRISPR-Cas nucleases in human cells. Nature biotechnology 31, 822-826.
Gratz, S. J., Cummings, A. M., Nguyen, J. N., Hamm, D. C., Donohue, L. K., Harrison, M. M., Wildonger, J., and O'Connor-Giles, K. M. (2013). Genome engineering of Drosophila with the CRISPR RNA-guided Cas9 nuclease. Genetics 194, 1029-1035.
Haurwitz, R. E., Jinek, M., Wiedenheft, B., Zhou, K., and Doudna, J. A. (2010). Sequence- and structure-specific RNA processing by a CRISPR endonuclease. Science (New York, N.Y. 329, 1355-1358.
Hsu, P. D., Scott, D. A., Weinstein, J. A., Ran, F. A., Konermann, S., Agarwala, V., Li, Y., Fine, E. J., Wu, X., Shalem, O., et al. (2013). DNA targeting specificity of RNA-guided Cas9 nucleases. Nature biotechnology 31, 827-832.
Hutvagner, G., and Zamore, P. D. (2002). A microRNA in a multiple-turnover RNAi enzyme complex. Science (New York, N.Y. 297, 2056-2060.
Hwang, W. Y., Fu, Y., Reyon, D., Maeder, M. L., Tsai, S. Q., Sander, J. D., Peterson, R. T., Yeh, J. R., and Joung, J. K. (2013). Efficient genome editing in zebrafish using a CRISPR-Cas system. Nature biotechnology 31, 227-229.
Jinek, M., Chylinski, K., Fonfara, I., Hauer, M., Doudna, J. A., and Charpentier, E. (2012). A Programmable Dual-RNA-Guided DNA Endonuclease in Adaptive Bacterial Immunity. Science (New York, N.Y.
Kamioka, Y., Sumiyama, K., Mizuno, R., Sakai, Y., Hirata, E., Kiyokawa, E., and Matsuda, M. (2012). Live imaging of protein kinase activities in transgenic mice expressing FRET biosensors. Cell structure and function 37, 65-73.
Kawai, T., and Akira, S. (2007). Antiviral signaling through pattern recognition receptors. Journal of biochemistry 141, 137-145.
Kormann, M. S., Hasenpusch, G., Aneja, M. K., Nica, G., Flemmer, A. W., Herber-Jonat, S., Huppmann, M., Mays, L. E., Illenyi, M., Schams, A., et al. (2011). Expression of therapeutic proteins after delivery of chemically modified mRNA in mice. Nature biotechnology 29, 154-157.
Li, J. F., Norville, J. E., Aach, J., McCormack, M., Zhang, D., Bush, J., Church, G. M., and Sheen, J. (2013). Multiplex and homologous recombination-mediated genome editing in Arabidopsis and Nicotiana benthamiana using guide RNA and Cas9. Nature biotechnology 31, 688-691.
Mali, P., Yang, L., Esvelt, K. M., Aach, J., Guell, M., DiCarlo, J. E., Norville, J. E., and Church, G. M. (2013). RNA-guided human genome engineering via Cas9. Science (New York, N.Y. 339, 823-826.
Miller, J. C., Tan, S., Qiao, G., Barlow, K. A., Wang, J., Xia, D. F., Meng, X., Paschon, D. E., Leung, E., Hinkley, S. J., et al. (2011). A TALE nuclease architecture for efficient genome editing. Nature biotechnology 29, 143-148.
Ngo, V. N., Davis, R. E., Lamy, L., Yu, X., Zhao, H., Lenz, G., Lam, L. T., Dave, S., Yang, L., Powell, J., et al. (2006). A loss-of-function RNA interference screen for molecular targets in cancer. Nature 441, 106-110.
Pattanayak, V., Ramirez, C. L., Joung, J. K., and Liu, D. R. (2011). Revealing off-target cleavage specificities of zinc-finger nucleases by in vitro selection. Nature methods 8, 765-770.
Qi, L. S., Larson, M. H., Gilbert, L. A., Doudna, J. A., Weissman, J. S., Arkin, A. P., and Lim, W. A. (2013). Repurposing CRISPR as an RNA-Guided Platform for Sequence-Specific Control of Gene Expression. Cell 152, 1173-1183.
Ramirez, C. L., Foley, J. E., Wright, D. A., Muller-Lerch, F., Rahman, S. H., Cornu, T. I., Winfrey, R. J., Sander, J. D., Fu, F., Townsend, J. A., et al. (2008). Unexpected failure rates for modular assembly of engineered zinc fingers. Nature methods 5, 374-375.
Randall, R. E., and Goodbourn, S. (2008). Interferons and viruses: an interplay between induction, signalling, antiviral responses and virus countermeasures. The Journal of general virology 89, 1-47.
Reyon, D., Tsai, S. Q., Khayter, C., Foden, J. A., Sander, J. D., and Joung, J. K. (2012). FLASH assembly of TALENs for high-throughput genome editing. Nature biotechnology 30, 460-465.
Shen, B., Zhang, J., Wu, H., Wang, J., Ma, K., Li, Z., Zhang, X., Zhang, P., and Huang, X. (2013). Generation of gene-modified mice via Cas9/RNA-mediated gene targeting. Cell research 23, 720-723.
Storici, F., Bebenek, K., Kunkel, T. A., Gordenin, D. A., and Resnick, M. A. (2007). RNA-templated DNA repair. Nature 447, 338-341.
Umov, F. D., Miller, J. C., Lee, Y. L., Beausejour, C. M., Rock, J. M., Augustus, S., Jamieson, A. C., Porteus, M. H., Gregory, P. D., and Holmes, M. C. (2005). Highly efficient endogenous human gene correction using designed zinc-finger nucleases. Nature 435, 646-651.
Wang, R., Teng, C., Spangler, J., Wang, J., Huang, F., and Guo, Y. L. (2013). Mouse Embryonic Stem Cells Have Underdeveloped Antiviral Mechanisms That Can Be Exploited for the Development of mRNA-mediated Gene Expression Strategy. Stem cells and development.
Warren, L., Manos, P. D., Ahfeldt, T., Loh, Y. H., Li, H., Lau, F., Ebina, W., Mandal, P. K., Smith, Z. D., Meissner, A., et al. (2010). Highly efficient reprogramming to pluripotency and directed differentiation of human cells with synthetic modified mRNA. Cell stem cell 7, 618-630.
Warren, L., Ni, Y., Wang, J., and Guo, X. (2012). Feeder-free derivation of human induced pluripotent stem cells with messenger RNA. Scientific reports 2, 657.
Whitehurst, A. W., Bodemann, B. O., Cardenas, J., Ferguson, D., Girard, L., Peyton, M., Minna, J. D., Michnoff, C., Hao, W., Roth, M. G., et al. (2007). Synthetic lethal screen identification of chemosensitizer loci in cancer cells. Nature 446, 815-819.

All references cited in the present application are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the Smaller ranges, and are also encompassed within the invention, Subject to any specifically excluded limit in the stated range. Where the stated range includes one or both of the limits, ranges excluding either or both of those included limits are also included in the invention.

A number of embodiments of the invention have been described. Nevertheless, it will be understood that various modifications may be made without departing from the spirit and scope of the invention. Accordingly, other embodiments are within the scope of the following claims.

1: A method for genome editing that uses a combination of synthetic mRNA that encodes Cas9 enzyme and sgRNA.

2. The method of claim 1, wherein the mRNA that encodes Cas9 and sgRNA contains a 5′diguanosine cap and poly(A) tail.

3: A method of claim 1, wherein a template to facilitate DNA break is additional provided.

4: A method of claim 3, wherein the template is a double-stranded DNA molecule.

5: A method of claim 2, wherein the template is a single-stranded DNA molecule.

6: A method of claim 2, wherein the template is an RNA molecule.

7: A method of claim 1, wherein Cas9 bears a mutation that disrupts one of the two endonuclease active site, SEQ ID NO:2, or SEQ ID NO:3

8: A method of claim 1, wherein Cas9 bears a mutation that disrupts both endonuclease active sites, SEQ ID NO:4.

9: A method of claim 1, wherein Cas9 is fused to another enzyme that can alter epigenetic markers on either the DNA or chromatin proteins.

10: A method of claim 1, wherein Cas9 mRNA contains modified nucleotides.

11: A method of claim 1, wherein sgRNA contains modified nucleotides.

12. The method of claim 9 or 10, wherein the modified nucleotides comprise 5-methyl-Cytosine, 2-Thio-Uracil, or pseudouracil.

13: A method of claim 1, wherein the molar ratio between Cas9 mRNA:sgRNA is between 1:1,000 to 1,000:1.

14: A method of claim 1, multiple sgRNAs targeting different sites in combination with mRNA molecules encoding one or more different Cas9 enzymes from different species or bearing different mutations are introduced into the same cells.

15: A method of claim 2, wherein the repair template is localized to the DNA break site through fusion to sgRNA as on one molecule.

16: A method of claim 2, wherein the repair template is localized to the DNA break site through fusion to an aptamer that binds Cas9.

17. The method of claim 1, wherein the method also includes adding B18R.

18. A Cas9 protein that is non-naturally occurring and has a mutation that disrupts one of the two endonuclease active site, wherein said mutated Cas9 protein is encoded by the DNA of SEQ ID NO: 2.

19. A Cas9 protein that is non-naturally occurring and has a mutation that disrupts one of the two Cas9 endonuclease active sites, wherein said mutated Cas9 protein is encoded by the DNA of SEQ ID NO: 3.

20. A Cas9 protein that is non-naturally occurring and has a mutation that disrupts both CAS9 endonuclease active sites, wherein said mutated Cas9 protein is encoded by SEQ ID NO: 4.

21. A non-naturally occurring CRISPER-Cas system comprising an mRNA that encodes for a mutated Cas9 protein that has a mutation in its nuclease gene and at least one mRNA that encodes for a guide RNA that upon entry into a cell produces the mutated Cas9 protein and guide RNA and that targets and hybridizes to a target sequence of a DNA with a single point mutation that upon action of the mutate Cas9 protein and guide RNA corrects the mutation in the target sequence.

22. The method of claim 22, wherein the cas9 mRNA contains one or more modified nucleotides.

23. The method of claim 22, wherein the cas9 mRNA and guide mRNA is transfected into the cell.

24. The method of claim 24, wherein the transfection is done in the presence of B18R.

25. The Cas 9 variants as set forth according to SEQ ID NOS: 2-4.

26. An engineered, non-naturally occurring all RNA, vector free, viral free-CRISPR/Cas system for gene editing comprising the cas 9 variants of claim 25.

27. A method of altering expression of at least one gene product comprising introducing into a eukaryotic cell containing and expressing a DNA molecule having a target sequence and encoding the gene product an engineered, non-naturally occurring all RNA Clustered Regularly Interspaced Short Palindromic Repeats (CRISPR)—CRISPR associated (Cas) (CRISPR-Cas) system that is vector free and viral free.

28. The method of claim 19 further comprising the cas 9 variants of seq ID Nos: 2-4.

29. A kit comprising an engineered, programmable, non-naturally occurring all RNA CRISPR-Cas system comprising a Cas 9 protein as set forth in sequence ID Nos: 1-4, and instructions for use.
